(19) United States
(12) Patent Application Publication
(10) Pub. No.: US 2003/0191297 A1
(43) Pub. Date: Oct. 9, 2003

(54) AVIAN GHD GENES AND THEIR USE IN METHODS FOR SEX IDENTIFICATION IN BIRDS

(76) Inventors: RICHARD GRIFFITHS, GLASGOW (GB); BELA TIWARI, OXFORD (GB)

Correspondence Address: WENDEROTH LIND & PONACK, 2033 K STREET, NW,SUITE 800, WASHINGTON, DC, 20006

(21) Appl. No.: 08/973,363

(22) PCT Filed: Jun. 5, 1996
(86) PCT No.: PCT/GB96/01341

(30) Foreign Application Priority Data
Jun. 6, 1995 (GB) 9511439.3

(51) Int. Cl. A01K 67/027; C07H 21/04; C07K 16/00; C07K 14/00

(52) U.S. Cl. 536/23.1; 530/350; 530/387.1

(57) ABSTRACT

The invention provides for DNA sequences (introns and exons) encoding two genes located on the Z and W chromosomes of birds. These genes can be used to identify the sex of most birds (Class Aves). In addition, the genes control the sex of the birds, a genetic process which is initiated as an embryo. Nucleic acid fragments, proteins, polypeptides, antibodies and related products and their use in medicine and agriculture are provided. The invention may be used in sex identification or for controlling the sex of adults or the progeny of commercially important animals.

### INTRODUCTION

The present invention relates to proteins, polypeptides, nucleic acid fragments, antibodies and related products and to their use in medicine and agriculture, for instance in diagnosis and therapy. More particularly the invention relates to a gene or genes which can be used to ascertain the sex of avian adults, embryos, cells, and tissues. These genes also control the sex of birds starting with action in the embryos and so control the sex of the progeny of birds

Much of our understanding of sex determination comes from three, extensively studied, model systems. In two of these, the fruitfly Drosophila melanogaster and the nematode Caenorhabitis elegans, it is the ratio of X chromosomes to autosomes that initiates sexual differentiation (Hodgkin 1992). In the mouse a single gene, SRY, located on the Y chromosome provides the impetus for male development; a pattern that is thought to be conserved throughout the mammals (Koopman et al. 1991 Foster, et al. 1992).

At the genetical level these three species employ very different molecular mechanisms, not only to control sex determination itself but to accommodate the differing dosages of genes that result from the males possessing a single X and the female two X chromosomes. These basic differences are largely due to the independent evolution of the three mechanisms and strongly suggests that other means of sex determination will have evolved elsewhere in the animal kingdom.

One class in which little is known about sex determination is the birds. They exhibit female heterogamety which means that the female has Z and W sex chromosomes and the male ZZ. This immediately suggests that sex determination in this class has an independent origin to that of their sister class, the mammals where it is the male that is heterogametic. Furthermore, it has been shown that whilst female mammals inactivate one of their X chromosomes as a method of dosage compensation (Grant & Chapman 1988), this does not seem to be a device employed by birds (Baverstock et al. 1982).

However, similarities do exist between the birds and mammals. The W chromosome, like the Y chromosome is usually smaller than its partner, and is also characteristically heterochromatic in appearance (Christidis 1990). The main exceptions to this rule are found in the ‘primitive’ representatives of both classes: the monotremes and the ratites where the morphological differences between the sex chromosomes are poorly defined (Graves 1987, Tagaki et al. 1972).

The heterochromatization of the W and Y results from the replacement of functional genetic loci with ‘junk DNA’ sequences. This process is thought to be a consequence of a suppression of recombination that has arisen to ensure that genes vital to the development of the heterogametic sex remain linked on the Y or W chromosome (Charlesworth 1991). As a result only a few genes such as Ube1y (Kay et al. 1991, Mitchell et al. 1991), Zfy (Page et al. 1987) and SRY itself remain on the mammalian Y chromosome. A similar situation is thought to prevail on the avian W chromosome where the presence of any functional genes has yet to be demonstrated, although it does possess vast arrays of repetitive elements (Griffiths & Holland 1990, Tone et al. 1982).

A further similarity in sex determination in birds and mammals is that the development of the male phenotype appears crucially dependent on the appearance of the testis. The female phenotype is the result of the ‘default pathway’. For mammals this was first demonstrated by Jost (1947) who grafted an embroyonic testis into genetically female rabbit embryos prior to sex determination. This was sufficient to allow the development of functional males. The same experiment has been carried out on chick embryos with comparable results (Stoll et al. 1978).

Once the testis has formed, the process of masculinization is adopted by the testicular hormones. The genetical switch that initiates testis determination is known to be SRY in mammals (Koopman et al. 1991). In birds, there appears to be no SRY homologue on the W chromosome (Griffiths 1991), although this is unsurprising given the separate evolution of sex determination in the two classes.

The only other pertinent evidence on the genetics of avian sex determination come from reports of chickens with abnormal chromosome complements. Table 1 shows data from Crew (1954) and McCarrey and Abbott (1979) on the phenotypes of the aneuploids so far described. These results suggest that the presence of the W chromosome in the aneuploid AA ZZW and the polyploid AAA ZZW has not acted as a dominant determinant of the female phenotype. This may mean that sex in birds may be determined more by the autosome to Z ratio, as in Drosphila and C. elegans. However, a ZO aneuploid which could confirm this hypothesis has yet to be described.

It must also be born in mind that XXY kangaroos, where SRY is thought to be the key male determining switch, exhibit both male and female characteristics (Graves 1987). This suggests that the limited aneuploid data that is available for birds should be interpreted with some caution.

To conclude, the genetic mechanism that controls sex determination in birds has not yet been elucidated. Here we suggest that a gene we have termed CHD-W(Chromodomain-Helicase-DNA binding on the W chromosome) alone or acting in conjunction with a closely related gene CHD-1A (Chromodomain-Helicase-DNA binding 1 Avian) initiates female development in birds.

### THE INVENTION

It is believed that all birds such as chickens and other species of commercial significance, will have two or more genes of the CHD type which will have a nucleotide sequence similar to the nucleotide sequences shown in FIG. 5, FIG. 7 and FIG. 8 and that the gene products will be proteins which are crucial to the determination of the sex of the organism. One of these genes will be located on the W chromosome and the other on an autosome or Z chromosome.

It will be understood that the exact sequence of the two genes will vary between species and between individuals of the same species at least at the nucleotide level and often also at the protein level. Complete or partial sequences of the chicken genes are shown in FIG. 5, FIG. 7 and FIG. 8. The gene or protein which contains sequence corresponding to those in FIG. 5, FIG. 7 and FIG. 8 will hereafter be referred to as an CHD-gene and proteins and fragments thereof, polypeptides, nucleic acids and fragments thereof and oligonucleotides containing part of a CHD gene will hereafter be referred to as CHD-proteins, CHD-nucleic acids and so on.

The present invention therefore provides a CHD-protein or a fragment thereof or polypeptide comprising a CHD-gene or a part thereof, subject to the proviso below.

The present invention also provides a protein or a fragment thereof or a polypeptide containing a mimetope of an epitope of a CHD-protein or fragment thereof of polypeptide containing a CHD-gene or a part thereof, subject to the proviso below. Such proteins, fragments and polypeptides are hereafter referred to as CHD-mimetope proteins or fragments thereof and CHD-mimetope polypeptides.

The present invention also provides a CHD-nucleic acid or a fragment thereof or oligonucleotide comprising a CHD-gene, or a part thereof subject to the proviso below.

In a particular aspect the present invention provides a single or double stranded nucleic acid comprising the CHD-gene of a bird or a part thereof of at least 17 contiguous nucleotide bases or base pairs, or a single or double stranded nucleic acid hybridizable with the CHD-gene of a bird, or part thereof of at least 17 contiguous nucleotide bases or base pairs, subject to the proviso below.

The invention further provides a nucleic acid or fragment thereof or an oligonucleotide encoding a CHD-protein or fragment thereof or a polypeptide comprising a CHD-gene or a part thereof or a CHD-mimetope protein or a fragment thereof or CHD-mimetope polypeptide, subject to the following proviso. These nucleic acids, fragments and oligonucleotides may have sequences differing from the sequences of CHD-nucleic acids, fragments and oligonucleotides due to alternative codon usage and/or encoding alternative amino acids sequences or mimetopes.

The present invention does not, however extend to any known protein or fragment thereof or polypeptide or nucleic acid or fragment thereof or oligonucleotide containing a CHD-gene related sequence such as the Saccharomyces cedvisiae SNF2/SWI2 gene, Drosophila polycomb and HP1 genes described below, insofar as that protein or fragment, polypeptide, nucleic acid or fragment or oligonucleotide is known per se.

The amino acid sequence of the CHD-gene has similarities to the chromobox and Helicase motifs of a number of discovered genes known to be involved in the remodelling of chromatin. This suggests that the CHD-protein of the present invention may have a regulatory function involving chromatin remodelling. However, none of these genes contain the chromobox and the Helicase of the CHD-gene which are conserved in conjunction, at least in the chicken, great tit, mouse and yeast but are not conserved in conjunction in the sequences of chromatin remodelling proteins not associated with sex determination at least at the stage of testis formation in birds. A gene that produces a protein having chromatin remodelling capacity but lacking these characteristic motifs is therefore outside the scope of the present invention.

In addition there are certain residues in the amino acid sequence of the chromobox and those residues immediately downstream thereof, of the CHD-gene which are also conserved at least between those found in the chicken, great tit, mouse and yeast but are not conserved in the sequences of chromatin remodelling proteins not associated with sex determination at least at the stage of testis formation in birds. Any one of these conserved residues is therefore considered characteristic of the CHD-gene proteins of the present invention. The characteristics of a CHD-chromobox will give a more complete and comprehensive description of the CHD-chromobox which can also be considered characteristic of the CHD-gene proteins of the present invention. A protein having chromatin remodelling capacity and a helicase motif but originating from a gene that lacks all or most of these characteristic amino acid residues in the chromobox motif is therefore outside the scope of the present invention.

The characteristic amino acids residues are shown in the alignment in FIG. 11, which is described in more detail below. When aligned with the illustrated sequences as shown, these residues fall at positions, 11, 12, 20, 27, 34 inside the chromobox and 3, 6, 8, 12-15, 16 immediately downstream.

The nucleotide base sequence of the CHD-gene includes bases which encode the chromobox and Helicase motifs of chromatin remodelling proteins as described above. However, the base sequence of the CHD-nucleic acids of the gene will include codons specifying both or either chromobox and Helicase motifs and the former will have codons specifying one or more of the characteristic amino acid residues described above and/or will be hybridizable with a sequence that controls the sex determination of birds under conditions which substantially prevent hybridization to other sequences in birds that do not have these characteristics.

Preferably the CHD-nucleic acids of the invention encode a chromobox and a helicase and one or more, preferably all, of the characteristic chromobox amino acid residues and meet the above hybridization requirements.

Fragments of CHD-nucleic acids according to the present invention will likewise contain codons specifying the chromobox and helicase motifs or including at least part of either of these motifs or CHD-gene adjacent to the codons encoding these features and/or will be hybridizable with a sequence that controls the sex determination of birds under conditions which substantially prevent hybridization to other sequences in birds that do not have these characteristics.

Oligonucleotides containing the CHD-gene or a part thereof according to the present invention may contain codons specifying the chromobox or helicase motifs or including at least part of these motifs or CHD-gene but this is not essential. However all such oligonucleotides of the invention must be capable of hybridizing with a sequence or sequences that control the sex determination of birds or a gene intron, preferably under conditions which substantially prevent hybridization with any sequence not associated with sex determining sequence.

A sex determining sequence referred to herein is a sequence which contains the CHD-gene and which encodes a factor which when expressed at the appropriate stage and level during embryo development may result in testis formation and subsequent growth of the embryo as a male. It may alternatively refer to a sequence which encodes a factor which when expressed at the appropriate stage and level during embryo development prevents testis formation and results in the subsequent growth of the embryo as a female.

The hybridization conditions referred to above which prevent unwanted hybridization with sequences not associated with the sex determining gene will depend to some extent on the length of the nucleic acid, fragment or oligonucleotide of the invention tested. Thus for instance lower stringency will be sufficient to secure hybridization to sequences associated with the sex determining gene whilst preventing unwanted hybridization when the nucleic acid or fragments several thousand nucleotide base pairs in length than for a fragment of only a few hundreds of bases or an oligonucleotide of from 17 bases up to a few tens or hundreds of bases. With the smallest oligonucleotides and fragments of the invention hybridization conditions will be such that only complete complementarity between the oligonucleotide and or fragment and the sequences associated with the sex determining gene will result in hybridization.

Preferred nucleic acids and fragments of the invention will only hybridize selectively to the sequences associated with the sex determining gene or genes under conditions requiring at least 80%, for instance 85, 90 or even 95% more preferably 99% complementarity. Yet more preferred nucleic acids and fragments of the invention are those having a sequence corresponding exactly to that of those illustrated in FIG. 5, FIG. 7 and FIG. 8 although the nucleotide sequences by be longer or shorter than those illustrated and or may contain normally intronic sequences associated with these sequences

The invention particularly provides an oligonucleotide, polypeptide, nucleic acid or protein comprising the entire sequence of the CHD-gene of a bird and more preferably comprising the entire amino acid or nucleotide sequence of the chicken as set out in any one of FIGS. 1, 3, 5, 7, 8, 9, 10, 11.

The nucleic acids hybridizable with the CHD-gene of a bird are preferably hybridizable under moderate, or more preferably, high stringency conditions as defined below:

| | |
|---|---|
| Moderate stringency: | |
| Buffer: | 2 × SSC |
| Temp: | 50° C. |
| Annealing period: | 6-8 hrs |
| High stringency: | |
| Buffer: | 1 × SSC |
| Temp: | 65° C. |
| annealing period: | 6-8 hrs |

Moderate stringency as defined above corresponds with about 75% homology. High stringency as defined above corresponds with about 90% homology. 1×SSC is 0.15 M sodium chloride, 0.015 M sodium citrate, pH 7.0.

Preferably the portion of the nucleic acid corresponding to or hybridizable with the CHD-gene is at least 20, more preferably at least 30, 40 or 60 and most preferably 100 or more nucleotide bases in length.

The nucleotide strands of the invention may be single or double stranded DNA or RNA. DNA's of the invention may comprise coding and/or non-coding sequences and/or transcriptional and or translational start and/or stop signals and/or regulatory, signal and/or control sequences such as promoters, enhancers and/or polyadenylation sites, endonuclease restriction sites and/or splice donor and/or acceptor, in addition to the CHD-gene sequence. Included within the DNA's of the invention are genomic DNA's and complementary DNA's (cDNA's) including functional genes or at least an exon containing the CHD-gene. They may also contain non-coding sequences such as one or more introns. Single stranded DNA may be the transcribed strand or the non-transcribed (complementary) strand. The nucleic acids may be present in a vector, for instance a cloning or expression vector, such as a plasmid or cosmid or a viral genomic nucleic acid. RNA's of the invention include unprocessed and processed transcripts of DNA, messenger RNA (mRNA) containing the CHD-gene and anti-sense RNA containing a sequence complementary to the CHD-gene.

Nucleic acids of the present invention are particularly useful as primers for polymerase chain reactions (PCRs) conducted to ascertain the sex of a bird as defined below. They may also be used to express proteins or fragments or polypeptides corresponding to the whole or a part of a CHD-protein (whether or not containing a CHD-gene) or as probes in hybridization experiments. As used herein the term “fragments” used in connection with proteins is intended to refer to both chemically produced and recombinant portions of proteins.

The CHD-proteins and fragments thereof and polypeptides containing the CHD-gene or a part thereof and CHD-mimetope proteins and fragments thereof and CHD-mimeotope polypeptides of the invention are useful in immunodiagnostic testing and for raising antibodies such as monoclonal antibodies for such uses. Antibodies against such proteins and fragments and polypeptides as well as fragments of such antibodies (which antibody fragments include at least one antigen binding site) including chemically derived and recombinant fragments of such antibodies, and cells, such as eukaryotic cells, for instance hybridomas and prokaryotic recombinant cells capable of expressing and, preferably secreting antibodies or fragments thereof against such proteins or fragments, also form part of the present invention.

The nucleic acids of the invention may be obtained by conventional means such as by the recovery from organisms using PCR technology or hybridization probes, by de novo synthesis or a combination thereof, by cloning the CHD-nucleic acids described below or a fragment thereof or by other techniques well known in the art of recombinant DNA technology.

Proteins and fragments thereof and polypeptides of the invention may be recovered from cells of organisms expressing a CHD-gene or generated by expression of a CHD-gene or coding sequence contained in a nucleic acid of the present invention in an appropriate expression system and host, or obtained by de novo synthesis or a combination thereof, by techniques well known in the art of recombinant DNA technology. The proteins, fragments thereof and polypeptides of the invention will contain naturally occurring L-a-amino acids and may also contain one or more non-naturally occurring a-amino acids having the D- or L-configuration

Antibodies may be obtained by immunization of a suitable host animal and recovery of the antibodies, by culture of antibody producing cells obtained from suitably immunized host animals or by in vitro stimulation of B-cells with a suitable CHD-protein, fragment or polypeptide or CHD-mimetope, protein, fragment or polypeptide and culture of the cells. Such cells may be immortalized as necessary for instance by fusion with myeloma cells. Antibody fragments may be obtained by well known chemical and biotechnological methods.

All these techniques are well known to practitioners of the arts of biotechnology. Reference may particularly be made to the well known text book “Molecular cloning: A laboratory manual” 2nd Edition (Eds Sambrook, J., Fritsch, E. F. and Maniatis, T.), (Cold Spring Harbour Laboratory, New York, 1989), hereafter referred to as “Maniatis”.

The invention further provides the use of a nucleic acid, protein, polypeptide, antibody, or antibody producing cell as hereinbefore defined including the SNF2/SWI2, polycomb and HP1 or other chromobox or helicase containing protein for ascertaining the sex of a cell or organism of a bird or for isolating nucleic acids useful in ascertaining the sex of a bird and for instituting single sex breeding programmes.

Knowledge of the chicken or great tit sex determining gene or genes can be used to isolate the equivalent gene or genes from other birds. Once isolated from a particular species, this gene or genes and its sequence can typically be used in two types of application:

1. The construction of sequence based sexing tests which can be applied to embryos, tissues and other biological materials containing nucleic acids.

2. The genetic modification of the germ line of birds to create breeding systems that produce offspring statistically biased towards one sex or of one sex only (single sex breeding systems).

A particularly preferred technique for ascertaining the sex of a bird in accordance with the invention involves the use of an oligonucleotides as primers in a PCR, for instance as follows:

A cell or cells or remains thereof are obtained, for instance by surgical removal from an embryo or from the quill of a feather, and the DNA is released by a crude lysis procedure for instance using a detergent or by heating. Primer olignucleotides of the invention are used to initiate a conventional PCR in order to amplify W chromosome linked CHD-related DNA from the cells. The products of the PCR are analysed by agarose gel electrophoresis and detected using labelled probes or by visual inspection. The presence of amplified CHD-W DNA indicates the presence of a CHD-W gene in the cells and thus, in birds, that the cell(s) were female. An example of a similar technique has been carried out by Griffiths & Tiwari (1995) on the Spix's Macaw (Cyanopsitta spixii). This is the world rarest bird (Guiness Book of Records) and DNA obtained from a moulted feather was sufficient to allow nested PCR amplification with CHD primers to show the bird was a male.

This technique may be applied for instance to identify the sex of embryos or adults for subsequent breeding programs in other bird species, or to control the sex of the progeny of breeding stock for commercial exploitation (by selection of the breeding stock or by slaughter or termination of animals of undesired sex).

The oligonucleotide primers for ascertaining or controlling sex in one species may also be used to ascertain or control sex in another species since hybridization of the primers to the CHD-gene of the other species will still serve to amplify the species-specific sequences.

Techniques for conducting such determinations are well known in the art of recombinant DNA technology.

In another aspect the present invention provides a process for isolating a W-chromosome specific sequence associated with the CHD-W gene of a bird which comprises probing a genomic library from a female of the species preferably of W chromosome sequences, for instance of lambda phage, cosmid or YAC library or cDNA library constructed from a tissue expressing the gene, with a probe comprising a nucleic acid, fragment or oligonucleotide of the invention as hereinbefore defined and a detectable label under high or moderate stringency.

Using the newly isolated subclone, Southern blots are performed on male and female DNA of the species of interest at high stringency to confirm that the correct clone has been isolated. The CHD-gene probe should give a female specific signal (other male/female shared bands may also be present at lesser intensities). The subclone is sequenced using standard methods and primers suitable for PCR chosen from the sequence so identified.

Alternatively, other approaches to cloning the sequences related to the sex determining gene could be used such as PCR methods using “degenerate” oligonucleotides. (For methods in PCR see, for example, “PCR Protocols—a Guide to Methods and Application”; edited by M. A. Innis, D. H. Gelfand, J. J. Sninsky, T. J. White; published by Academic Press, Inc.).

Preferably the probe is CHD-1A or CHD-W or a fragment thereof or a nucleic acid or fragment or oligonucleotide having a sequence exactly as set out in FIG. 5, FIG. 7 or FIG. 8 for the chicken. Techniques for forming a genomic or cDNA library and for probing and detecting the detectable label and isolating the nucleic acid identified by the probe are well known in the art of biotechnology and recombinant DNA manipulation. The process may be conducted for instance using a probe having the chicken sequence such as the CHD-W sequence to identify and isolate the corresponding sequence from another bird such as Turkey. The thus-identified sequence can then be used to generate primers for PCR which in turn can be used to ascertain the sex of an individual or of cells, tissues, embryos or ovaries of the bird. This technique has been used by obtaining DNA from the Chicken and Hyacinth Macaw (Anodorhynchus hyacinthinus) to design primers for the Spix's Macaw (Griffiths & Tiwari 1995). This will permit experiments to ascertain sex to be conducted and controlled sex breeding of the bird as described below.

In addition, the nucleotide sequence of the CHD-genes are sufficiently conserved so that CHD primers can be designed that will allow PCR in a range of bird species. The primers P1, P2 and P3 shown in FIG. 14 will allow CHD-W and CHD-1A amplification in a range of birds that allows sex to be identified.

The isolated nucleic acid, fragment or oligonucleotide may thereafter be amplified, cloned or sub-cloned as necessary. The invention further provides a process for detecting the sex of an individual bird or of cells, tissues, embryos, foetuses or ovaries or a bird, comprising conducting a polymerase chain reaction using DNA from the individual, cell, tissue, embryo or ovary as template and a nucleic acid, fragment or oligonucleotide of the invention as primer. Preferably the nucleic acid, fragment or oligonucleotide of the invention used as primer is CHD-W or CHD-1A or a part thereof and has a sequence corresponding exactly to the chicken sequence in FIG. 5, FIG. 7 or FIG. 8 or a part thereof or is a nucleic acid, fragment or oligonucleotide which is a W-chromosome specific sequence associated with the sex determining gene or genes of a bird of the same species as the individual cell, tissue, embryo, foetus or ovary whose sex is to be ascertained. The W-chromosome specific sequence associated with the sex determining gene or genes of the bird involved may itself have been obtained by the process of isolation and amplification or cloning described above. It can also be obtained by deduction from the sequence in FIG. 5, FIG. 7 or FIG. 8 or a sequence from another bird or animal.

The identification of the sex determining gene or genes according to the present invention raises the possibility of controlling the sex of progeny of commercially important animals such as chickens, turkeys and other avians. This will be valuable in many aspects of animal breeding and husbandry such as where one sex has more desirable characteristics, for instance only female progeny are desired for egg-laying breeds of chicken. The economic advantages of single sex breeding programmes and strategies for instituting these are described for instance in “Exploiting New Technologies in Animal Breeding; Genetic Developments”, (Eds. Smith, C., King, J. Q. B. and McKay, J. C.), (Oxford University Press, Oxford, 1986).

The nucleic acids making up all or part of the sex determining gene, from the same or different animal species, can be introduced into any early embryo through established transgenic technology. This latter includes microinjection of DNA into pronuclei or nuclei of early embryos, the use of retroviral vectors with either early embryos or embryonic stem cells, or any transformation technique, (including microinjection, electroporation or carrier techniques) into embryonic stem cells or other cells able to give rise to functional germ cells. These procedures will allow the derivation of individual transgenic animals (founder transgenics) or chimeric animals composed in part of cells carrying the introduced DNA. Where the functional germ cells of the founder transgenic or chimeric animal carry the introduced DNA it will be possible to obtain transmission of the introduced DNA to offspring and to generate lines or strains of animals carrying these DNA sequences.

The nucleic acids making up part or all of the coding sequence of the sex determining gene, or derivatives of it, may be introduced in combination with its own regulatory sequences (promoter/enhancers etc.) or regulatory sequences from another gene, the whole making the “construct”, to give expression from the construct at an appropriate developmental stage and tissue location critical to sex determination in the bird species under consideration. For example, in the chicken this would be between 6 and 7 days post lay.

### MATERIALS AND METHODS

Isolation of pGT-W, pGT1.7 and pGT8 Great Tit Clones

A great tit (Parus major) library was constructed from genomic DNA, partially restricted with MboI, and the IFixII vector (Stratagene). The library was screened at high stringency with the 724 bp probe (GT-W) cloned from a W chromosome specific polymerase chain reaction (PCR) product derived from the great tit (Griffiths & Tiwari 1993). Positive plaques were subject to two rounds of purification. Clone IGT2 contained an insert of 9.6 kb that hybridized strongly to the probe sequence. The insert was subcloned as two EcoRI fragments of 1.7 kb (pGT1.7) and 8 kb (pGT8) into EcoRI cut pT7/T3 (Pharmacia).

Isolation of CHD Genes from the Chicken

Two chicken cDNA libraries were screened. The first was a mixed sex chick stage 10-12 cDNA library in IZapII which had been reamplified on 2 occasions This library was provided by Dr I. J. Mason. The second library was constructed from mixed sex, 10 day chick mRNA. Total RNA was extracted using a guanidine thiocyanate based technique (Koopman 1993) and mRNA isolated using a Promega PolyATtract system 1000. A IZapII library was constructed using a Stratagene ZAP-cDNA synthesis kit. Plaques (2×10⁵) from the stage 10-12 day library were screened at moderate stringency with a subcloned 433 bp HindIII/SacI fragment from pGT8 that contained the 123 bp region with identity to the mouse CHD-1 gene (Delmas et al. 1993). A similar number of plaques from both libraries were screened with bases 428-4428 of CHD-1A (see FIG. 5). The 10 day library was also screened with bases 4059-5303 of CHD-1A (see FIG. 5). Positive plaques were purified prior to the excision of pBluescript plasmids and cloned inserts insert from IZapII using techniques recommended by Stratagene.

Sequencing

All sequencing was carried out using the T7 DNA polymerase/7-deaza-dGTP chain termination sequencing kit from USB. All sequencing unless otherwise specified was carried out in both directions either by subcloning or through exonuclease III deletion with the Promega Erase-a-Base system.

Southern Blot Analysis and Hybridization

Genomic DNA was extracted from blood (Griffiths & Holland 1990), digested with the appropriate restriction enzyme and Southern blotted onto Zeta-Probe GT under neutral conditions as described by the manufacturer (Bio-Rad). Prehybridizations and hybridizations were carried out in 0.25M Na₂HPO₃/5% SDS at either 65° C. (high stringency) or 62° C. (moderate stringency). Subsequent washes were carried out for a total of 1 hour in three changes of either 0.5×SSC (75 mM NaCl/7.5 mM sodium citrate (pH7.5))/0.1% SDS at 65° C. (high stringency) or 1×SSC/0.1% SDS at 45° C. (low stringency).

Sex Identification with PCR on Dried and Limited DNA in a Spix's Macaw

Stratagene provided a genomic Hyacinth Macaw Lambda FixII Library (Cat. No. 946402). Plaques were screened at moderate stringency with a 1.3 Kb Chicken CHD-W subclone (spans 2670-4003 nucleotides in the related Mouse CHD1 gene (Delmas et al., 1993)). A CHD-W genomic fragment was isolated and aligned to the chicken and mouse homologues to allow the design and construction of 3 primers (5′ to 3′)

P3 AGATATTCCGGATCTGATAGTGA,

P2 TCTGCATCGCTAAATCCTTT and

P1 ATATTCTGGATCTGATAGTGA(C/T)TC.

DNA from the wild Spix's Macaw was extracted (Thomas & P\ddot{aa}bo 1993) from 1 cm portions of the tips of 3 moulted flight feathers collected in 1994 and 1995. The negative extraction control was taken through an identical procedure. 1.5% of these extraction products or 50 ng of genomic DNA from the reference samples were subject to semi-nested PCR. Primary amplification consisted of 20 cycles with primers P3 and P2; 1% of the primary PCR product was subject to 30 cycles of amplification with P2 and P1. Samples were denatured for 1.5 min at 95° C. then cycled between 57° C./30 sec, 72° C./15 sec and 94° C./30 sec with a 5 min final extension. Products were precipitated, cut with DdeI, reprecipitated and electrophoresed through visigel separation matrix (Stratagene). The accuracy of the test was confirmed using DNA from Spix's and Hyacinth Macaws of known sex (n=5 p=0.03). Uncut secondary PCR product from the wild bird was isolated (Dretzen et al. 1981), cloned using the Stratagene pCR-Script SK(+) kit and sequenced to confirm that the product had originated from a Spix's Macaw

Sex Identification with PCR in a Variety of Birds

DNA was isolated from blood taken from Chicken (5 individuals used), Marsh Harrier (28; Circus aeruginosus) and Kestrel (18 Falco tinninculus) all sexed by adult plumage, Bee-eater (4; Merops apiaster; plumage/behaviour), Boobook Owl (2; Ninox novaesiae), White-faced Owl (2; Ptilopsis leuctis) Burrowing Owl (2; Speotyto cumcularia), Eurasian Eagle Owl (2; Bubo bubo), Long-eared Owl (2; Asio otus), Tawny Owl (3; Strix aluco, adult size), Starling (5; Stumus vulgaris; Beak colour) and African Marsh Warbler (5; Acrocephalus baeticatus; reproductive behaviour). DNA from a variety of parrots sexed by laparotomy was also used: Blue Fronted Amazon (3; Amazona a aestiva), Orange Winged Amazon (5; Amazona amazonica), Red Lored Amazon (3; Amazona autumnalis), Yellow Crowned Amazon (2; Amazona o ochrocephala), Tucamen Amazon (2; Amazona tucamana), Blue and Gold Macaw (6; Ara ararauna), Citron Crested Cockatoo (2; Cacatua sulphurea citronocristate), Lesser patagonian (2; Cyanolisous patagonus), Blue Headed Pionus (1; Pionus menstruus), Plum Headed Parakeet (4; Psittacula cyanocephala), African Grey Parrot (12; Psittacus erithacus), Blue Throated Conure (2; Pyrrhura cruentata), Senegal Parrot (3; Seneglus poicephalus).

All the birds listed above were sexed from DNA using exactly the same PCR reaction. PCR reaction volumes of 20 μl were made up of Promega Taq buffer (1× is 50 mM KCl, 10 mM Tris.HCl, 1.5 mM MgCl₂, 0.1% Triton X-100), 200 μM of each dNTP, P2 (5′-TCTGCATCGCTAAATCCTTT) and P3 (5′-AGATATTCCGGATCTGATA) primers (approx 1 μM), 50-200 ng of genomic DNA and 0.15 units of Taq polymerase. The thermal treatment was 94° C./1.5 mins followed by 30 cycles of 55 or 56° C./15 sec, 72° C./15 sec, and 94° C./30 sec with a finish of 56° C./1 min and 72° C./5 min. HaeIII (5 units; Promega) was used to cut 8 ul of PCR product in 1× Promega restriction enzyme buffer 3 and 50 ng/μl bovine serum albumin (Sigma) in a total volume of 10 μl. The digests and uncut PCR product were precipitated before being electrophoresed in a visigel (Stratagene) with ethidium bromide (40 ng/ml) at 3.5 V/cm.

### RESULTS

The plasmid pGT-W contains a 724 bp insert that hybridizes to a 4.9 kb fragment only in the female great tit. Its DNA sequence was determined (FIG. 1) and contains a 457 bp open reading frame. A search of the EMBL DNA and protein sequence database found no significant matches. The sequence does contain a simple sequence consisting of a 22 bp run of thymidines.

The pGT-W insert was used to probe Southern blots, at low stringency, of PvuII restricted genomic DNA of male and female great tit, starling, jarkdaw (Corvus monedula), pied wagtail (Motacilla alba) and a species of new world flycatcher. These are species that cover the extremes of the passeriforme order according to the recent phylogeny of Sibley et al. (1988). In all but the jackdaw convincing hybridization to a single female specific fragment could be observed. In all species, hybridization to one or more non-sex specific fragments was also shown. A similar experiment was carried out with a non-passerine, the bee-eater (Merops apiaster), and this too resulted in faint hybridization to a female specific fragment and two, somewhat stronger bands, in both sexes.

In order to further investigate the nature of the pGT-W insert we attempted to clone a larger fragment of genomic DNA which incorporated this motif. From around 1.5×10⁵ plaques from a great tit genomic library, two positives were obtained. After purification one of these gave superior hybridization and was investigated further. The 9.7 kb insert was subcloned as pGT1.7 and pGT8 containing 1.7 kb and 8 kb respectively. The pGT1.7 was sequenced in its entirety and approximately 2.8 kb of the sequence of pGT8 was determined. Both were sequenced in a single direction. A 723 bp region, starting 133 bp from the 5′ end of pGT8 had a sequence that corresponded exactly to the pGT-W insert (FIG. 2).

The sequences derived from these subclones were used to search the EMBL database using the FASTA algorithms (GCG, Wisconsin package vers 7.3). A region of 123 bp, starting 994 bp from the 5′ end of pGT8, showed a 79% nucleotide sequence identity to bases 3855-3977 of the mouse CHD-1 gene (FIG. 3; Delmas et al. 1993). This corresponds to an 88% identity at the amino acid level.

Southern blots of PvuII digests of genomic DNA from male and female chicken and lesser black-backed gull (Larus fuscus) were probed at low stringency with a 433 bp SacI/HindIII fragment of pGT8 that included the 123 bp region with CHD-1 identity (FIG. 4). FIG. 12 shows that in the chicken hybridization was with a fragment of 3.1 kb in the female only and with fragments of 1.5 and 6.0 kb in both sexes. In the gull hybridization is similarly with a female specific fragment of 4.0 kb a fragment of 3.0 kb in males and females.

Delmas et al., (1993) have already demonstrated the universal occurrence of the CHD-1 in the mammals. The evidence this blot provides, which features species representing both the major divisions of the birds, suggests that a minimum of two types of CHD gene exist in this Class. The first we termed CHD-W to denote its W linkage. The 123 bp region from the great tit would appear to be a short exon from this gene. The second hypothetical gene is closely related to CHD-W and we have it termed CHD-1A, where the A denotes its avian nature. This gene is either Z or autosomally linked as it occurs in both sexes.

Isolation of CHD-1A

The SacI/HindIII great tit probe was used at low stringency to screen a IZapII cDNA library from stage 10-12 (33-49 hrs after the appearance of the primitive streak) chicken embryos. A plating of 2×10⁵ plaques yielded a panel of 25 positive clones, 19 of these continued to hybridize intensely after purification. From three clones Z4, Z6 and Z11 a composite 6608 nucleotide sequence (FIG. 5) was determined using the strategy illustrated in FIG. 6.

The insert from the Z6 clone (bases 418-4426; FIG. 5) and a BgIII (AGATCT) fragment of the Z4 clone (bases 4059-5303; FIG. 5) were used separately to screen a similar number of plaques from a second cDNA library constructed from 10 day old chicken embryos. This screening identified a total of 45 positives of which 16 were found to have sequence identity with the composite sequence derived from the first library. Two additional clones contained a closely related sequence that is dealt with below.

A proportion of the clones from both libraries show variation from the sequence given in FIG. 5 in one respect. Clones Z1, Z13, Z17, Z20 and Z23 are identical to the composite sequence 5′ to base 4327 from there they terminate in an additional 37 to 163 bases of a new sequence that is identical in all five. Two clones from the second library CC43 and CC56 have 22 or 254 bp of the same sequence at their 5′ ends. Downstream of this motif both clones regained homology with the composite sequence at base 4328 and show no further deviation from the original sequence. From these seven clones a composite 264 bp sequence can be derived and this is illustrated in FIG. 7. None of the seven clones contain the whole of this sequence. Moreover, none of the ten clones that span the 4327/4328 insertion point contain any of this additional region. If inserted at this position, the motif has an in frame, open reading frame spanning its entire length. The motif is extremely adenosine rich and this makes the amino acid lysine extremely common in the putative translation (see FIG. 7). There are no splice donor or acceptor sites within the motif suggesting it is a final rather than an intermediary product of splicing.

Hybridization of a probe running from 2534 to 4428 bp of the sequence chicken sequence to a blot of PvuII cut, male and female chicken genomic DNA shows that hybridization occurs to fragments that are both W and autosomally or Z chromosomally located. The level of hybridization is significantly stronger to the fragments common to both sexes suggesting that the probe represents the CHD-1A gene.

CHD-1A is very closely related to the mouse CHD-1 gene being 79.8% identical in a 5152 nt overlap. At the amino acid level the identity is raised to 90% over 1750 residues. We do have an additional 1202 bp of the 3′ untranslated region but have not encountered a clone with an AATAAA termination signal or a 3′ homopolymeric T tail. Both mouse and chicken sequences contain a stop codon in the same relative positions and sequence similarity is insignificant after this point. The published mouse sequence does not contain the additional 264 bp motif described above.

The database search also identified an unpublished chicken derived sequence tagged as a delta crystallin binding protein (DCBP), with even greater identity than the mouse CHD-1 gene: 99% over 2293 bp and 94% over 571 amino acid residues. The DCBP sequence is of 2292 bp which extends over nucleotides 1922 to 4214 of CHD-1A (FIG. 5). Despite the high nucleotide sequence identity the region of amino acid similarity does not extend the full length of the DCBP. This is due to apparent deletions in the DCBP clone that provides an initiation methionine codon (257 nt DCBP) and a stop codon (1939 nt DCBP). The extremely high sequence identity, the fact that identity is maintained after the apparent stop in the DCBP sequence, that none of the 41 CHD-related clones we found have exact sequence identity and that only small sequencing mistakes would be required to introduce false stop and start codons suggests that the DCBP sequence is CHD-1A but has been sequenced slightly inaccurately. Further evidence is required to confirm this.

The database search with the whole CHD-1A gene also revealed significant identity to a previously unidentified portion of a 15 kb region of S. cerivisiae chromosome V. This region comprises an open reading frame of 4.4 kb which lies between the RAD4 (Gietz & Prakash 1988) and the poly-A binding protein (Sachs et al. 1986) gene coding regions. In an overlap of 1538 amino acids, the whole of the yeast open reading frame, there is an identity of 37.7% and a similarity of 59% (FIG. 10). The degree of conservation this similarity implies suggests the yeast sequence encodes a homologue of CHD-1A that we shall term CHD-1Y for the sake of discussion.

Delmas et al., (1993) identified four motifs in CHD-1 with possible functional significance. CHD-1A retains such close homology to CHD-1 that these regions are virtually unchanged and are likely to perform similar functions as they do in the mouse.

The first motif is a chromodomain (Paro & Hogness 1991) which falls between residues 274 and 311 (FIG. 9). FIG. 11 compares the amino sequence of this region to that of eight others identified through a search of the EMBL database. The sequences fall into three categories. The first comprises the domain from CHD-1, CHD-1A and CHD-1Y. The second and third chromobox groups have been previously identified by (Pearce et al. 1992). The HP1 class comprises the Drosophila (James & Elgin 1986) and human (Saunders et al. 1993) HP1 genes and two murine modifier (Mod) genes (Singh et al. 1991). The HP1 class is characterized mainly by glutamic acid rich block of six residues upstream of the chromobox. The third group, the Pc class, comprises the Drosophila Pc gene (Paro & Hogness 1991) itself and its putative murine homologue the Mod3 gene (Pearce et al. 1992).

A search of the EMBL data base with the CHD-1A putative helicase domain (residues 451-911, FIG. 9) raises the identity between this and CHD-1Y to 55% in an overlap of 471 amino acids. There is also significant, but lesser identity to, the putative helicase motifs in the human (Okabe et al. 1992), and S. cerivisiae (Laurent et al. 1992) SNF2 gene, human (Muchardt & Yaniv 1993) and Drosophila Brahma (Tamkun et al. 1992), S. cerivisiae NPS1/STH1 (Laurent et al. 1992, Tsuchiya et al. 1992), human excision repair protein ECCR6 (Troelstra et al. 1992) and the RAD54 (Emery et al. 1991) and MOT1 (Davis et al. 1992) genes of S. cerivisiae. It should be noted that none of these latter genes contain a chromobox.

Only the four CHD genes show significant homology to the third motif, a DNA binding region identified by Delmas et al.,(1993), whilst only CHD-1A and CHD-1 have the three short basic HSDHR motif near the carboxy terminus, although this region is yet to be sequenced in CHD-W. The CHD-1Y gene apparently terminates before this point so does not share this motif. An extended discussion of the homology of the mouse CHD-1 gene can be found in (Stokes & Perry 1995).

Isolation of CHD-W

Two, CC14 and CC4, of eight CHD-1 related clones isolated from the 10 day chick embyro library using 349-4359 nt of CHD-1A as a probe, overlap (FIG. 5) to provide the 1316 bp of sequence given in FIG. 8. This is a sequence closely related to, but distinct from CHD-1A. Identity over the 1316 bp overlap is 90.5% and 90.1% at the nucleotide and amino acid level respectively. An alignment of the putative translations of CHD-1, CHD-1A and CHD-W is given in FIG. 9. The amino acid identity between CHD-1 and CHD-1A at 93.4% is marginally lower than that between that of CHD-1 and CHD-W, 94.2%, over the same region

The 1335 bp insert of CC4 was used at moderate stringency to probe a male/female, PvuII cut genomic blot featuring mouse, ostrich (Struthio camelus), chicken, bee-eater and hyacinth macaw (FIG. 13). Hybridization with the mouse and ostrich shows no evidence of any sex linkage, bands of the same size and equal intensity appearing in both sexes. Hybridization with the ostrich is particularly strong, greater even than with the cognate sequence in the chicken. This suggests that the genome size of the ostrich is considerably smaller than that of the chicken. It also demonstrates that the CHD genes cannot be used to sex the ostrich and, it is suggested, the other members of the ratites. There is no evidence from further work (reported later) that this effect should occur in other Parvclasses of the birds (Sibley et al. 1988).

In all the bird species apart from the ostrich, hybridization occurs with two types of fragment some that are female unique and others that are shared between the sexes. In the chicken some of the latter are of the same size as those hybridizing with the CHD-1A probe and result from cross hybridization under the conditions of low stringency that we employed. When probed with the CC4 sequence it is clear that hybridization with the female linked fragments is far stronger, at least in the chicken than with the shared fragments (bear in mind, also, that the female chicken only has a single dosage of the W linked gene). This indicates that CC4 is W linked and represents part of CHD-W.

The DNA contained in the Southern blot of the male and female chickens probed in FIG. 13 contained identical amounts of DNA. However, examination shows that the shared bands are twice as strong in males (ZZ) as they are in females (WZ). The only way this could have happened is if the CHD-1A gene is Z linked. It is suggested this is the case in all birds.

Sex Identification with PCR on Dried and Limited DNA in a Spix's Macaw

The first test was devised to sex DNA extracted from the feathers of the last wild Spix's Macaw. This was the rarest bird on the planet and needed to be sexed so a mate could be selected from the 31 captive birds that remained. The test presented two problems. The first was extracting DNA from feathers the second providing a test that would work.

The procedure was published in Griffiths & Tiwari (1995) which covers the extraction of the DNA. The second test was to provide DNA from a Hyacinth Macaw which would yield data to allow construction of primers. A IFIX II library was provided by Stratagene and this was probed with the insert of the CHD-1A clone Z6 (-227-5302 FIG. 6) at moderate stringency. This provided 7 positive clones (A1, A2, A7, A8, A13, 1.2 and 5C). The inserts were extracted cut with MboI and subcloned into the BamI cut pUC18. This sublibrary was probed again with the Z6 insert but this time at high stringency. The A12.3 subclone hybridized. This was sequenced and contained 111 bp which is aligned to the chicken and mouse CHD genes in FIG. 14. The similarity of this fragment to the chicken CHD-W suggested this was the Hyacinth Macaw homologue of the W chromosome located gene.

The data from A12.3 supplied information for the design of the primers required. It also provided evidence that the CHD sequences were sufficiently conserved in this region that a single set of primers could be designed to amplify both genes. Three primers, P1, P2 and P3, were designed to allow seminested PCR (FIG. 14). This technique allowed amplification of a 104 bp region of both CHD-W and CHD-1A from DNA that was available from two captive Spix's Macaws of known sex. In each sex the PCR products were of the same size but sequence determination revealed that the CHD-W derived PCR product possessed a DdeI restriction enzyme site which was lacking in the CHD-1A product. Thus PCR amplification and DdeI cleavage of male Spix's Macaw DNA yields a only single product of 104 base pairs (bp), whilst from female DNA two products are apparent, one of 104 bp and one of 73 bp. The presence of the CHD-1A product in both sexes acts as a control to ensure the PCR amplification has been successful (FIGS. 15 & 16).

DNA was extracted from feathers moulted by the wild Spix's Macaw using a technique devised for the purification of ancient DNA (Thomas & P\ddot{aa}bo 1993). The PCR-based test described above was used to demonstrate that CHD-W was not present in the sample (see FIG. 16). This confirmed that the wild bird is male. A female Spix's macaw was released in March 1995 as a prospective mate.

Sex Identification with PCR on a Variety of Birds

Birds can be sexed from DNA by showing the presence (female: ZW) or absence (male: ZZ) of the female specific W chromosome. At the molecular level this is carried out by the recognition of a W-linked marker. This can only be done after a W chromosome DNA marker is identified in the avian species. The test developed for the Spix's Macaw used CHD-W as a W linked marker. The data collected in designing this test suggested that this method may work to sex a variety of birds.

If the same test is to work on other bird species then two criteria must be met. The first is whether the PCR primers will amplify both CHD genes in other bird species. The Spix's Macaw test used the tiny amounts of DNA extracted from feathers so a seminested PCR was required. This used 3 primers which are aligned to the Mouse and Chicken CHD nucleotide sequences in FIG. 14. The primer sites are highly conserved, there is no difference between the chicken genes and a solitary difference between the Mouse and Chicken in the 5′ region of the P2 site. Theoretically, the primers should anneal to other bird species and, if a reasonable amount of DNA is available (>50 ng), a single pair of primers should provide sufficient amplification.

A second requirement for the test is that the PCR products can be separated using a restriction endonuclease. In the Spix's Macaw the DdeI enzyme cuts CHD-W but not CHD-1A. FIG. 14 shows that this discrimination would also occur in the Chicken. However, the DdeI cutting site CTNAG is not present in the CHD-1A of Spix's Macaw (CTNGG) nor the Chicken (CANAG) for different reasons. This suggests that the DdeI sit is open to mutation so this form of discrimination is unlikely to be conserved. Other discriminatory sites are available: DdeI and MaeII sites are unique to CHD-W and the HaeIII, MboII and XhoI sites to CHD-1A and can be considered the first option If these fail the CHD-W and CHD-1A PCR fragments can be cloned and sequenced so discriminatory sites can be discovered.

The theory we have presented suggests that a sexing test based on both avian CHD genes should work on many other bird species. Does this work in practice? The birds selected for trial are from across the avian class: Chicken (5 individuals), Marbled Murrelet (18), Kestrel (8), Marsh Harrier (28), Bee-eater (4), 1 pair of six species of Strigidae Owls from different genera (see Methods), Starling (5) and African Marsh Warbler (5).

The primers amplify a PCR product of the predicted size in all of the birds using primers P2 and P3 on 50-100 ng of genomic DNA extracted from blood. FIG. 17 illustrates this for 3 bird species but also includes amplification from human DNA. This shows that tests using P2 and P3 are open to human DNA contamination so appropriate precautions must be taken.

The HaeIII restriction enzyme cut the CHD-1A fragment alone in all 13 species (FIG. 17) and, from the sequence data, would also have worked on the Spix's Macaw (FIG. 16). FIG. 17 shows that the CHD-1A in males is cut into two fragments (45 bp, 59 bp) which are not easily visible on the gel. In females CHD-W is uncut by Haelli so remains at 104 bp. The discrimination using HaeIII provided correct sex identification in all individuals.

### DISCUSSION

The CHD Genes

The female specific great tit probe GT-W was described by Griffiths and Tiwari (1993) as a means of identifying sex in this species. The results presented here suggest this sequence represents part of a intron in a W linked gene. By moving downstream from this sequence it has been possible to isolate a putative exon from a gene that we have named CHD-W due to its close sequence identity to the mouse CHD-1 gene (Delmas et al. 1993) and its W location.

Using the CHD-W fragment we attempted to isolate a similar, W linked sequence that Southern blot analysis had shown was present in the chicken. From several clones a 6606 bp cDNA sequence was assembled but although it has close sequence identity to the great tit CHD-W fragment Southern blot analysis shows it is not located on the W chromosome. This second gene was termed CHD-1A (A=avian). This blot shows a second gene closely related to CHD-1A is W located. This sequence could not be cloned from a stage 10-12 chick cDNA library although 19 CHD-1A clones were isolated. However, two clones yielding 1347 bp of a second CHD gene were isolated along with a further 14 CHD-1A clones from a day 10 chick cDNA library. Southern blot analysis showed that this second clone was W chromosome derived and so represents CHD-W. Attempts are underway to isolate the remainder of CHD-W.

Southern blots of a variety of bird species showed that CHD-W is W chromosome linked in all birds except the ostrich. This suggests that the gene is sex linked throughout the class with the exception of the primitive ratites, which the ostrich represents, where it appears to be autosomally located.

An alternative explanation is that the CHD-W is in fact W linked in ratites but occurs in a region of the W chromosome which still recombines with the Z chromosome. If CHD-1A were Z linked, then recombination between Z and W linked copies of CHD would maintain their sequence identity resulting in the apparently autosomal location indicated by the Southern blot. A mammalian example would be the MIC2 and STS genes that are located in the pseudoautosomal region of the Y chromosome (Ellis & Goodfellow 1989) and would give analogous results to those observed here.

Two lines of evidence support this alternative hypothesis. The first is that the Southern blot analysis suggests that CHD-1A is Z linked in non-ratites which would make the chromosomal location of the CHD-genes consistent throughout the class. Hybridization of CHD-1A to genomic blots is apparently stronger to fragments from male birds which would result from this sex having two copies of any Z linked gene in comparison to a single copy in the female (this result is not clear cut and requires confirmation by chromosomal in situ). The second line of evidence is that the sex chromosomes of the ratites are not morphologically differentiated as is the case with other birds (Christidis 1990). Morphological similarity suggests recombination still occurs between extensive regions of the ratite Z and W which may include the CHD genes and so produce the pattern of hybridization observed.

Although we have yet to clone the whole of CHD-1A the 6606 bp sequenced so far shows a close identity to the mouse CHD-1 gene over the putative coding region. It also includes all four features identified by Delmas et al. (1993) as having possible functional significance. This includes a chromodomain, a helicase, a DNA binding motif and a basic, five amino acid motif that is repeated three times (FIG. 9). The similarity of the sequence derived thus far from CHD-W to that of CHD-1 and CHD-1A suggest it will be of similar length and possess these same motifs. We have also identified an alternatively spliced form of CHD-1A and CHD-W which has a similar adenine rich motif inserted at an identical point (4327/4328 nt CHD-1A and 1316 nt CHD-W). The exact form of these alternative mRNAs is yet been elucidated. It is interesting to note that we obtained no clones that spanned these breakpoints which contained this additional motif; the sequence was built up from partial sequences derived from either 5′ or 3′ terminii of different clones. Delmas et al., (1993) produced a mRNA Northern blot probed with fragments of CHD-1 occurring 5′ to this breakpoint and discovered an mRNA species of about 4 kb. This would correspond to a species cleaved near this insertion point. What purpose this would serve is unknown. Moreover the putative yeast homologue of CHD, CHD-1Y, which was identified from amino acid identity to CHD-1A from the genomic sequence on the EMBL database does not apparently have a similar motif. This is suggested because the CHD-1Y sequence was derived from a genomic clone which would allow the identification of any such sequence were it to be spliced in the normal manner.

The significance of the four functional domains found in the CHD genes will be discussed in turn. The first, the carboxy-terminal trimer repeat of five basic amino acid residues, has no known function arid is not shared by any other sequences from the EMBL database. Furthermore, the CHD-1Y gene which is truncated by a little over 200 amino acid residues in comparison to CHD-1 and CHD-1A does not contain this motif.

The second functional domain was identified by Delmas et al. (1993) as having sequence selective DNA binding capacity. Whether this is highly specific or just to A+T rich regions was not established. They also noted that this domain contains Lys-Arg-Pro-Lys-Lys and Arg-Gly-Arg-Pro-Arg motifs which enable genes like HMG-1, D1 and Engrailed to bind in the minor groove of A+T rich DNA.

A third functional motif is located towards the N-terminus of the CHD-protein and is termed the chromodomain [Chromatin Organization Modifier; Paro, 1990 #459]. This is a highly conserved domain of between 37-50 amino acids that has been shown to be represented in the genomes of plants, nematodes, insects and vertebrates (Singh et al. 1991). Several chromobox genes have been isolated from human, mouse and Drosophila and have been divided into the polycomb (Pc) class and the heterochromatin protein-1 (HP1) class on the basis of related structure (Pearce et al. 1992)). The CHD-genes have a distinct form of the chromobox characterized by close homology between yeast and vertebrate forms in the 5′ half of the box itself but extending a further 17 residues downstream. These differences indicate that this form of the chromobox defines a third subgroup the CHD class

The Pc gene forms one of a eponymously named group (Pc-g) of about 12 genes defined through homeotic mutants in Drosophila that prevent fixation and maintenance of a determined state. They act as transcriptional repressors of homeotic genes, notably of the antennapedia complex (ANT-C; Paro, 1990). Members of the ANT-C and the other major group of Drosophila homeotic genes, the bithorax complex (BX-C), are responsible for defining segmental identity during development (Kaufman et al. 1980, Lewis 1978). Initially, their expression patterns are designated by early acting maternal and segmentation genes (see 4,6,7 kennison). However, these maternal genes are only transiently expressed. During the later stages of development their role as transcriptional activators is adopted by an assemblage of genes including the trithorax group (Trx-g), whilst many of their repressive effects are assumed by the Pc-g (Kennison 1993).

The polycomb (Pc) gene itself is perhaps the best studied member of the Pc-g. Zink and Paro (1989) used Pc-B-galactose fusion proteins to show that it binds to around 100 different sites on the polytene chromosome including loci where other members of the Pc-g are located. Any disruption of the chromodomain abolishes the specificity of this reaction (Messmer et al. 1992). However, the Pc-g protein appears to lack any type of endogenous DNA binding capacity so it is thought that it acts as part of a protein complex with other components that are responsible for the site specific DNA binding (Paro 1990).

The repressive effects of the Pc-g are thought to be the result of chromatin compaction. In other words, the DNA is packaged into heterochromatin to prevent or reduce the expression of functional genes (Paro 1990). This is a mechanism related to position effect variegation (PEV; (Henikoff 1990)), to dosage compensation in mammals which sees the complete heterochromatization of one of the female's X chromosomes and possibly to gene imprinting whereby the expression of maternally and paternally inherited alleles differs (Peterson & Sapienza 1993). The links with PEV have recently been substantiated in that HP1, a recognized modifier of PEV, and Pc both contain chromodomains (Paro & Hogness 1991). Like the Pc protein, HP1 appears to form part of a structural complex that transforms euchromatin to heterochromatin. Furthermore, both PEV and the repressive effects of Pc are passed, in a clonal manner, to daughter cells ((Henikoff 1990, Struhl 1981); a characteristic also of gene imprinting.

With the CHD-type gene containing both a DNA binding motif and a chromobox it may appear reasonable to suggest that they encode repressors with an endogenous, site selective DNA binding system. However, CHD genes contain a further functional motif that is structurally related to the Helicases. The sequence identity is closest to the yeast SNF2/SWI2 (Abrams et al. 1986) and Drosophila Brahma genes (Tamkun et al. 1992), both of which are transcriptional activators. Indeed, Brahma is part of the Trx-g which are considered direct antagonists to the Pc-g. Other genes which contain more distantly related Helicase domains are involved in DNA repair and chromatid separation during mitosis (Laurent et al. 1993, Sung et al. 1993).

The SWI2 gene product has been shown to enhance the transcription of other genes probably as part of a complex that includes SWI1, SWI3, SNF5, SNF6 and in conjunction with gene specific DNA binding proteins (Laurent et al. 1991, Peterson & Heskowitz 1992). A mode of action strikingly similar to that of Pc.

Although it remains to be formally demonstrated that SWI2 is a helicase, it does have close structural similarities with proven Helicase genes and also possesses the required DNA stimulated ATPase activity (Laurent et al. 1993). Laurent et al., go on to postulate that the SWI2 containing complex may act by two mechanisms acting either separately or in conjunction. In the first they envisage helicase mediated DNA melting to allow the egress of RNA polymerase II. Alternatively SWI2 could allow chromatin remodelling, in effect overcoming any inhibitory packaging of the DNA and so enhancing transcription.

The juxtaposition of a Helicase and a chromodomain within the same gene presents a paradox that may challenge the perceived roles of the two motifs. A simple explanation is that alternative splicing could remove one or other of these domains prior to translation. However, there is little support for this idea from the work of ourselves or Delmas et al., (1993).

An alternative explanation could be due to our lack of real knowledge about the function of the chromobox. Whilst it is well established that Helicases do disassociate DNA and so facilitate transcription (Matson & Kaiser-Rogers 1990), the role of the chromodomain in repression is based on more circumstantial evidence. Pc, as we have seen, does not bind DNA itself although mutations in the chromobox prevent the formation of site specific complexes. It is possible that the chromodomain is involved more in maintaining the structural integrity of the repressive complex than in the repressive mechanism itself. Based on this supposition, the CHD-protein may form a different type of complex able to bind at a site dictated or influenced by its own binding domain and activate these loci via helicase activity.

While both this scenario is speculative it is probable that CHD-type genes are active during development and are able to bring about heritable changes in transcription. The presence of an endogenous DNA binding domain suggests it has fewer targets than Pc, for example, which could form part of several different active complexes. With CHD-W being confined to the W chromosome is likely to have a role in some aspect of female development and we suggest this may be critical to the determination of gender. In support this hypothesis we were unable to find any CHD-W clones in a library constructed prior to sex determination which occurs at day 7 (Lutz-Ostertag 1954) but were able to isolate two clones from a smaller pool of candidates at day 10. This suggests that the expression of CHD-Y may occur at a time consistent with its having a sex determining role.

If CHD-W alone or in conjunction with CHD-1A causes sex determination in birds then several potential mechanisms are plausible.

(1) In the simplest scenario CHD-Y may act as a simple trigger like SRY (Koopman 1993) to either cause expression or repression of downstream genes in order initiate testis development.

(2) CHD-W may interact with other autosomal or Z linked genes whereby the dosage of CHD-W in comparison these other factors causes initiates development down the male or female pathways.

A more complicated scenario is if CHD-W acts in together with CHD-1A to cause sexual differentiation. Different mechanisms could operate depending whether CHD-1A turns out to be Z linked as we suspect or autosomal.

(3) If CHD-1A is Z linked, then male birds get two doses of the CHD-1A expression product to one in female birds. Perhaps the 1:1 ratio of functionally distinct CHD-1A and CHD-W products is what initiates female development whilst a double dosage of CHD-1A results in males.

(4) Alternatively, just the single dosage of Z linked CHD-1A product could result in female development and expression of CHD-W only occurs after sexual differentiation to equalize dosages of functionally similar proteins.

(5) If CHD-1A is autosomal however, it could be envisaged that CHD-1A and CHD-W are functional homologues and the three doses in females (AAW) is required to promote female development, whilst the double dosage in males (AA) causes the differentiation of the testis and the development of the male phenoype.

The evidence from aneuploid chickens discussed in the introduction, does suggest that the mechanism that does operate involves some degree of dosage dependence which tends to exclude mechanism (1). However the similarity of CHD-W to HP1, the Pc protein and other transcriptional modifiers that act through chromatin remodelling show that the expression of this type is crucially dependent on dosage (Locke et al. 1988). With the different dosages of gene product and/or potential target sites that aneuploids possess it may be that analysis of these type of mutants has, thus far, served to confuse the issue.

Sex Identification

The first W-chromosome linked DNA was isolated by Tone et al. (1982) from the Chicken. Since then, a number of other W-linked avian sequences have been discovered (e.g. Griffiths, 1990; Rabenold, 1991; Griffiths, 1993). In all but one case, described later, these DNA fragments appear to be non-functional repeats. For instance, the related XhoI and EcoRI fragments in Chicken may comprise 70-90% of the W chromosome (Saitoh et al. 1991). This repeat and others in the Lesser Black-backed Gull (Larus fuscus) can be used to sex birds by the rapid dot blotting technique (Griffiths & Holland 1990). Other less repetitive W chromosome markers can be used to sex birds either by probing Southern blots (Rabenold et al. 1991) or through the use of PCR (Griffiths & Tiwari 1993).

The major problem with all non-functional W-linked DNA is the speed with which they evolve. The chicken XhoI repeat is fairy typical. Through low stringency hybridization to a Southern blot it can be used to sex the Turkey (Meleagris gallopavo) and the Pheasant (Phasianus versicolor; Saitoh et al. 1991). These bird species are closely related to the Chicken by being members of the family Phasianidae. By contrast, the functional CHD-W region described here is 96% (3/67 FIG. 3) identical between Chicken and Spix's Macaw and this only drops to 86% between the Chicken CHD-W and the Mouse CHD1 (15/110 FIG. 3). This level of conservation means that the chicken CHD-W probe can be used on Southern blots to sex birds from all over the class Aves.

The only exception to the non-functional avian W-linked sequences is DZWM1 which is a putative gene, cloned from a cDNA turkey library. Like CHD-W this gene appears to be sex linked in many bird species. Unfortunately, so little information has been published in the papers that describe DZWM1 that the nature of the gene remains unknown (Dvorák et al. 1992, Halverson 1990, Halverson & Dvorák 1993).

For sexing large numbers of birds Southern blot analysis is slow and expensive. The technique that we have used is based on a PCR using P2 and P3 primers followed by a Haelli digestion of the of the amplified product. The digestion distinguishes between the CHD-W product which is uncut and the CHD-1A which is cut. The technique will work to sex a range of bird species that span the class Aves. The primers target a highly conserved region so are likely to be ‘universal’ to the birds but the discriminatory HaeIII site which cuts CHD-1A but not CHD-W shows no real reason to be conserved. If HaeIII does fail to be discriminatory other cutting sites have been suggested or the CHD-W and CHD-1A PCR products can easily be sequenced to look for an alternative. Alternatively, the different nucleotide sequence of the amplified CHD-W and CHD-1A suggests that the two PCR products would be separable on an agarose gel of around 3% or a non-denaturing acrylamide gel. This would remove the need for a cutting enzyme and may well make the sexing technique more easy to use.

The CHD based test appears to be fairly solid but the chances of a peculiar mutation in some bird species is not impossible. Cases concerning SRYISox3 genes on the sex chromosomes in mammals supports this claim. In two species of the vole Ellobius males have neither a Y chromosome nor an SRY gene (Just et al. 1995). In a second case, four species of Akodon, the Mole Vole, have 15-40% of fertile females with XY chromosomes and an SRY gene (Bianchi et al. 1993). These examples are particularly peculiar in that the SRY gene is accepted as the gene that determines sex throughout the mammals. In neither case would the detection of SRY reliably inform you of the animals sex.

These examples from the Muridae may never occur with the CHD genes of birds. However, it does suggest that sex identification by the amplification of CHD-W and CHD-NW should always be validated by a test on several individuals in a new species before it is applied. Despite this warning, the use of the test described here or by other means using the CHD-W or CHD1A, these genes provide a method to sex most bird species.

TABLE 1
Sex of domestic fowl with normal and abnormal chromosome
complements (from McCarrey & Abbott (1979) and Crew (1954)).

| Chromosome complement | Phenotype |
|---|---|
| AAZZ | Male |
| AAZW | Female |
| AZZW | Male? |
| AAZZZ | Male |
| AAAZZZ | Male |
| AAAZZW | Intersex/male |

### FIGURE LEGENDS

FIG. 1. The DNA sequence of the pGT-W insert.

FIG. 2. A map of the 9.6 kb insert of the IFixII clone isolated from the great tit using pGT-W. pGT1.7 and pGT8 are the two EcoRI subclones into which the fragment was divided. The broken line corresponds to the region with absolute sequence identity to the pGT-W insert. The position of the region with identity to the mouse CHD-1 gene is indicated.

FIG. 3. An alignment of 123 bp fragment of the great tit (GT) CHD-W gene in pGT8 with the autosomal/Z located chicken (C) CHD-1A the chicken CHD-W gene and bases 3855-3977 of the mouse (M) CHD-1 gene. An alignment of the deduced amino acid sequence is also given.

FIG. 4. The section of pGT8 that hybridized to a female specific fragment of 3.1 kb in the chicken. This probe was also used to screen the chicken cDNA library. The hatched line represents the female specific great tit motif shown in FIG. 3.

FIG. 5. The complete nucleotide sequence of CHD-1A as defined by the clones Z4, Z6 and Z11. Two asterisks underlie the position where part of the sequence illustrated in FIG. 7 is spliced onto the 5′ or 3′ ends of a proportion of the clones isolated. The ATG at nucleotide 228 is the start codon whilst TAA at 5388 is the stop codon.

FIG. 6. The strategies used to determine the nucleotide sequence of CHD-1A and CHD-W given in FIG. 5 and FIG. 8. The top line represents the mouse clone given by (Delmas et al. 1993). The three ‘Z’ clones of CHD-1A and the ‘CC4’ and ‘CC14’ clones of CHD-W were derived from either a stage 10-12 or a 10 day chick cDNA library respectively. Arrows indicate the direction of sequence determination. Note Z6 actually ran from −227 to 69. These nucleotides were determined and are found in FIG. 5

FIG. 7. A composite nucleotide sequence and putative translation of the motif that is found spliced to a proportion of the 5′ or 3′ terminii of CHD-1 clones or the 3′ end of the CHD-W clone CC14. The portion attached to the CC14 sequence is incomplete.

FIG. 8. A partial nucleotide sequence of CHD-W as defined by the clones CC4 and CC14.

FIG. 9. An alignment of the deduced amino acid sequences of the chicken (C) CHD-1A and CHD-W with the mouse (M) CHD-1. With gaps introduced to maximize alignment they show a sequence identity of 91.6% over 1365 residues. The $ sign indicates start and stop codons. Boxed sections are the chromodomain (C), Helicase (H), and the region containing the DNA binding domain (B) identified by Delmas et al., (1993). A trimer repeat of a basic HSDHR motif is underlined. A* denotes residue identity and. similarity.

FIG. 10. An alignment of the deduced amino acid sequences of CHD-1A and CHD-1Y a putative yeast homologue of the chicken gene identified through a search of the EMBL data base. With gaps introduced to maximize alignment they show a sequence identity of 37.7% over 1538 residues. | indicates identity and: conservative substitution.

FIG. 11. Comparison of 9 chromodomain sequences. Vertical lines indicate the extent of the chromodomain as defined by Paro & Hogness (1991). The top three sequences represent the CHD class of chromodomain to add to the HP1 class and Pc class][;−I08k9ouygytrdevz as defined by Pearce et al. (1992). The first letter of each annotation indicates the animal of origin: C, chicken; M mouse; D, Drosphila; H, human; Y, S. cerivisiae whilst the remainder identifies the gene type. The yeast gene is a possible CHD homologue identified by its close identity to the vertebrate forms. * indicates sequence identity within the groups and \hat{ } identity between all nine sequences. * indicate amino acid residues inside and downstream of the motif that are characteristic of the CHD class chromobox.

FIG. 12. Genomic Southern blots of DNA from male and female chickens and lesser black-backed gulls digested with PvuII and probed with a 433 bp HindIII/Sac fragment of pGT8 (FIG. 4.) at moderate stringency. Hybridization with female linked fragments and fragments common to both sexes can be observed in both species. Numbers give approximate sizes in kilobases.

FIG. 13. Genomic Southern blots of DNA from male (M) and female (F) mice, ostrich, chicken, bee-eater and hyacinth macaw probed with the 1335 bp insert of CC4 at moderate stringency. Hybridization with mouse and ostrich is with fragments shared by both sexes whilst the non-ratite birds show additional hybridization to female specific fragments. In these latter species, the signal from female linked hybrids is stronger than with autosomal/Z linked fragments indicating that the probe is derived from the W chromosome. Numbers give approximate sizes in kilobases.

FIG. 14. The nucleotide sequence of part of a single CHD1 gene isolated from the Mouse and the homologous genes from the Chicken, Hyacinth (A12.3 subclone) and Spix's Macaw all arranged as putative codons. Dashes denote nucleotides shared with the Mouse sequence. The primers designed are shown on the diagram. An arrow head indicates a non-synonymous mutation in the Spix CHD-W. The DdeI (CTNAG) and HaeIII (GGCC) sites are underlined.

FIG. 15. The technique of PCR sex identification in the Spix's Macaw. Semi-nested PCR amplification is carried out on both sexes with the primers P2/P3 then P1/P2 to provide products of identical sizes in both sexes. The products are then cut with restriction enzyme DdeI which cuts only the CHD-W product from the female. The cut products are run on a visigel and the difference between the sexes can be visually detected. See FIG. 17 for an example.

FIG. 16. DdeI restricted PCR products demonstrating that remaining wild Spix's Macaw is male. Lane 1. the wild bird 2. negative extraction control 3. known male 4. known female. The larger fragment is of 104 bp and the female W-chromosome specific fragment of 73 bp.

FIG. 17. Sex identification in the Marsh Harrier (MH), Chicken (C) and African Marsh Warbler (AMW) carried out using an identical reaction. For each species genomic DNA of male and female birds was subject to PCR with primers P2 and P3 and the product of 110 bp is visible in lanes 1 and 2. In lane 3 the entire male PCR product, amplified from CHD-1A, has cut into two parts with Haelll (65 bp, 45 bp). In females, lane 4 this HaeIII cut product is also present but the CHD-W product remains uncut so the sex can be identified. The ‘Kb’ lane contains a ‘1 Kb DNA ladder’ (BRL), the ‘H’ lane is PCR reaction with P2 and P3 carried out on human genomic DNA and −ve lane contains a negative PCR reaction.

### REFERENCES

Abrams, E., Neigeborn, L. & Carlson, M. 1986. Molecular analysis of SNF2 and SNF5, genes required for expression of glucose-repressible genes in Saccharomyces cerivisiae. Mol. Cell. Biol. 6: 3643-3651.

Baverstock, P. R., Adams, M., Polkinghorne, R. W. & Gelder, M. 1982. A sex-linked enzyme in birds—Z-chromosome conservation but no dosage compensation. Nature 296: 763-766.

Bianchi, N. O., Bianchi, M. S., Bailliet, G. & de la Chapelle, A. 1993. Characterization and sequencing of the sex determining region Y gene (SRY) in Akodon (Cricetiae) species with sex reversed females. Chromosoma 102: 389-395.

Charlesworth, B. 1991. The evolution of sex chromosomes. Science 251: 1030-1033.

Christidis, L. 1990. Animal cytogenetics 4: Chordata 3; B, Aves. Berlin, GebrŸder Borntraeger.

Crew, F. A. E. 1954. Sex determination. New York, John Wiley and Sons, Inc.

Davis, J. L., Kunisawa, R. & Thorner, J. 1992. A presumptive Helicase (MOT1 gene product) affects gene expression and is required for viability in the yeast Saccharomyces cerivisiae. Mol. Cell. Biol. 12:1879-1892.

Delmas, V., Stokes, D. G. & Perry, R. P. 1993. A mammalian DNA binding protein that contains a chromodomain and an SNF2/SWI2-like Helicase domain. Proc. Natl. Acad. Sci. USA 90: 2414-2418.

Dretzen, G., Bellard, M., Sassone-Corsi, P. & Chambon, P. 1981. A reliable method for the recovery of DNA fragments from agarose and acrylamide gels. Anal. Bichem. 112: 295-296.

Dvorák, J., Halverson, J. L., Gulick, P., Rauen, K. A., Abbott, U. K., Kelly, B. J. & Shultz, F. T. 1992. cDNA cloning of a Z- and W-linked gene in gallinaceous birds. J. Heredity 83: 22-25.

Ellis, N. & Goodfellow, P. N. 1989. The mammalian pseudoautosomal region. Trends Genet. 5: 406-410.

Emery, H. S., Schild, D., Kellogg, D. E. & Mortimer, R. K. 1991. Sequence of RAD54 a Saccharomyces cerivisiae gene involved in recombination and repair. Gene 104: 103-106.

Foster, J. W., Brennan, F. E., Hampikian, G. K., Goodfellow, P. N., Sinclair, A., Lovell-Badge, R., Selwood, L., Renfree, M. B., Cooper, D. W. & Graves, J. A. M. 1992. Evolution of sex determination and the Y chromosome: SRY-related sequences in marsupials. Nature 359: 531-533.

Gietz, R. D. & Prakash, S.1988. Cloning and nucleotide sequence analysis of the Saccharomyces cerevisae RAD4 gene required for excision repair of UV-damaged DNA. Gene 74: 535-541.

Grant, S. G. & Chapman, V. M. 1988. Mechanisms of X-chromosome regulation. Ann. Rev. Genet. 22: 199-233.

Graves, J. A. M. 1987. The evolution of mammalian sex chromosomes and dosage compensation: clues from marsupials and monotremes. Trends Genetics 3: 252-256.

Griffiths, R. 1991. The isolation of conserved DNA sequences related to the human sex-determining region Y gene from the lesser black-backed gull (Larus fuscus). Proc. R. Soc. Lond. B. 244: 123-128.

Griffiths, R. & Holland, P. W. H. 1990. A novel avian W chromosome DNA repeat sequence in the lesser black-backed gull (Larus fuscus). Chromosoma 99: 243-250.

Griffiths, R. & Kelsey, M. 1995. A mate for the last macaw. Daily Telegraph 43,544: 24.

Griffiths, R. & Tiwari, B. 1993. The isolation of molecular genetic markers for the identification of sex. Proc. Natl. Acad. Sci. USA 90: 8324-8326.

Griffiths, R. & Tiwari, B. 1995. Sex of the last wild Spix's macaw. Nature 375: 454.

Halverson, J. L. 1990. Avian sex identification by recombinant DNA technology. Proc. Assoc. Avian Vet. 256-262.

Halverson, J. L. & Dvorak, J. 1993. Genetic control of sex determination in birds and the potential for its manipulation. Poultry Sci. 72: 890-896.

Henikoff, S. 1990. Position-effect variegation after 60 years. Trends Genet. 6: 422-426.

Hodgkin, J. 1992. Genetic sex determination mechanisms and evolution. BioEssays 14: 253-261.

James, T. C. & Elgin, S. C. R. 1986. Identification of a nonhistone chromosomal protein associated with heterochromatin in Drosophila melanogaster and its gene. Mol. Cell. Biol. 6: 3862-3872.

Jost, A. 1947. Sur les effects de castration precoce de le embryo male du lapin. C. R. Soc. Biol. 141: 126-129.

Just, W., Rau, W., Vogel, W., Akhverdian, M., Fredga, K., Graves, J. A. M. & Lyapunova, E. 1995. Absence of SRY in species of the Vole Ellobius. Nature Genetics 11: 117-118.

Kaufman, T. C., Lewis, R. & Wakimoto, B. 1980. Cytogenetic analysis of chromosome 3 in Drosophila melanogaster: the homeotic gene complex in polytene chromosome interval 84A-B. Genetics 94: 115-133.

Kay, G. F., Ashworth, A., Penny, G. D., Dunlop, M., Swift, S., Brockdorff, N. & Rastan, S. 1991. A candidate spermatogenesis gene on the mouse Y chromosome is homologous to ubiquitin-activating enzyme. Nature 354: 486-489.

Kennison, J. A. 1993. Transcriptional activation of Drosophila homeotic genes fro distant regulatory elements. Trends Genet. 9: 75-79.

Koopman, P. 1993. Analysis of gene expression by reverse transcriptase—polymerase chain reaction. In: C. D. Stern & P. W. H. Holland (eds). Essential developmental biology: 233-242. Oxford, IRL Press.

Koopman, P., Gubbay, J., Vivian, N., Goodfellow, P. & Lovell-Badge, R. 1991. Male development of chromosomally female mice transgenic for SRY. Nature 351: 117-121.

Laurent, B. C., Treich, I. & Carlson, M. 1993. The yeast SNF2/SWI2 protein has DNA stimulated ATPase activity required for transcriptional activation. Denes & Dev. 7: 583-591.

Laurent, B. C., Treitel, M. A. & Carlson, M. 1991. Functional interdependence of the yeast SNF2, SNF5, and SNF6 proteins in transcriptional activation. Poc. Natl. Acad. Sci. USA 88: 2687-2691.

Laurent, B. C., Yang, X. & Carlson, M. 1992. An essential Saccharomyces cerevisiae gene homologous to SNF2 encodes a Helicase related gene. Mol. Cell. Biol. 12: 1893-1902.

Lewis, E. B. 1978. A gene complex controlling segmentation in Drosophila. Nature 276: 565-570.

Locke, J., Kotarski, M. A. & Tartof, K. D. 1988. Dosage-dependent modifiers of position effect variegation in Drosophila and a mass action model to explain their effects. Genetics 120: 181-190.

Lutz-Ostertag, Y. 1954. Contribution a l'etude canaux de muller chez l'embryon d'oiseaux. Bull. Biol. Fr. Belg. 88: 333-412.,

Matson, S. W. & Kaiser-Rogers, K. A. 1990. DNA helicases. A. Rev. Biochem. 59: 289-329., J. R. & Abbott, U. K. 1979. Mechanisms of genetic sex determination, gonadal sex differentiation, and germ-cell development in animals. Adv. Genet. 20: 217-290.

Messmer, S., Franke, A. & Paro, R. 1992. Analysis of the functional role of the Polycomb chromo domain in Drosophila melanogaster. Genes & Dev. 6: 1241-1254.

Mitchell, M. J., Woods, D. R., Tucker, P. K., Opp, J. S. & Bishop, C. E. 1991. Homology of a candidate spermatogenic gene from the mouse Y chromosome to the ubiquitin-activating enzyme E1. Nature 354: 483-486.

Muchardt, C. & Yaniv, M. 1993. A human homologue of Saccharomyces cerevisiae SNF2/SWI2 and Drosophila brm genes potentiates trascriptional activation by the glucocorticoid receptor. EMBO J. 12: 4279-4290.

Okabe, I., Bailey, L. C., Attree, O. F., Perkel, J. M., Nelson, D. L. & Nussbaum, R. L. 1992. Human and bovine homologues of Saccharomyces cervisiae SNF2/SWI2 a global activator of transcription in yeast. Nucl. Acids Res. 20: 4649-4655.

Page, D. C., Mosher, R., Simpson, E. M., Fisher, E. M. C., Mardon, G., Pollack, J., McGillivray, B., de la Chapelle, A. & Brown, L. G. 1987. The sex-determining region of the human Y chromosome encodes a finger protein. Cell 51: 1091-1104.

Paro, R. 1990. Imprinting a determined state into the chromatin of Drosophila. Trends Genet. 6: 416-421.

Paro, R. & Hogness, D. S. 1991. The polycomb protein shares a homologous domain with a heterochromatin-associated protein of Drosophila. Proc. Natl. Acad. Sci. USA 88: 263-267.

Pearce, J. J. H., Singh, P. B. & Gaunt, S. J. 1992. The mouse has a polycomb-like chromobox gene. Development 114: 921-929.

Peterson, C. L. & Heskowitz, 1.1992. Characterization of the yeast SWI1, SWI2, and SWI3 genes, which encode a global activator of transcription. Cell 68: 573-583.

Peterson, K. & Sapienza, C. 1993. Imprinting the genome: Imprinted genes, Imprinting genes, and a hypothesis for their interaction. Annu. Rev. Genet. 1993: 7-31.

Rabenold, P. P., Piper, W. H., Decker, M. D. & Minchella, D. J. 1991. Polymorphic minisatellite amplified on the avian W chromosome. Genome 34: 489-492.

Sachs, A. B., Bond, M. W. & Kornberg, R. D. 1986. A single gene from yeast for both nuclear and cytoplasmic polyadenylate-binding proteins: domain structure and expression. Cell 45: 827-835.

Saitoh, Y., Saitoh, H., Ohtomo, K. & Mizuno, S. 1991. Occupancy of the majority of DNA in the chicken W chromosome by bent repetitive DNA sequences. Chromosoma 101: 32-40.

Saunders, W. S., Chue, C., Goebi, M., Craig, C., Clark, R. F., Powers, J. A., Eissenburg, J. C., Elgin, S. C. R., Rothfield, N. F. & Earnshaw, W. C. 1993. Molecular cloning of a human homologue of Drosophila heterochromatin protein HP1 using anti-centromere autoantibodies with anti-chromo specificity. J. Cell Sci. 104: 573-582.

Sibley, C. G., Ahlquist, J. E. & Monroe, B. L. 1988. A classification of the living birds of the world based on DNA-DNA hybridization studies. Auk 105: 409-423.

Singh, P. B., Miller, J. R., Pearce, J., Kothary, R., Burton, R. D., Paro, R., James, T. C. & Gaunt, S. J. 1991. A sequence motif found in Drosophila heterochromatin protein is conserved in animals and plants. Nucl. Acids. Res. 19: 789-794.

Stokes, D. G. & Perry, R. P. 1995. The DNA-binding and chromatin-localization properties of CHD1. Mol. Cell Biol. 15: 2745-2753.

Stoll, R., Rashedi, M. & Maraud, R. 1978. Sur l'hormone de regression MŸllerrienne, agent inducteur do testicule chez l'embryon de poulet. Bull. Assist. Anant. 176: 131-143.

Struhl, G. 1981. A gene product required for correct initiation of segmetal determination in Drosophila. Nature 293: 36-41.

Sung, P., Bailly, V., Weber, C., Thompson, L. H., Prakash, L. & Prakash, S. 1993. Human Xeroderma pigmentosum group D gene encodes a DNA Helicase. Nature 365: 852-855.

Tagaki, N., Itoh, M. & Sasaki, M. 1972. Chromosome studies in four species of ratitae (Aves). Chromosoma 36: 281-291.

Tamkun, J. W., Deuring, R., Scott, M. P., Kissinger, M., Pattatucci, A. M., Kaufman, T. C. & Kennison, J. A. 1992. Brahma: a regulator of Drosophila hoeotic genes structurally related to the yeast transcriptional activator SNF2/SWI2. Cell 68: 561-572.

Thomas, W. K. & P\ddot{aa}bo, S. 1993. DNA sequences from old tissue remains. Methods Enzymol. 224: 406-419.

Tone, M., Nakano, N., Takao, E., Narisawa, S. & Mizuno, S. 1982. Demonstration of W chromosome-specific repetitive DNA sequences in the domestic fowl Gallus g. domesticus. Chromosoma 86: 551-69.

Troelstra, C., Van Gool, A., de Wit, J., Vermeulen, W., Bootsma, D. & Hoeijmakers, J. H. 1992. ERCC6, a member of a subfamily of putative helicases, is involved in Cockayne's syndrome and preferential repair of active genes. Cell 71: 939-953.

Tsuchiya, E., Uno, M., Kiguchi, A., Masouka, K., Kanemori, Y., Okabe, S. & Mikayawa, T. 1992. The Saccharomyces cerivisiae NPS1 gene, a novel CDC gene which encodes a 160 kDa nuclear protein involved in G2 phase control. Embo J. 11: 4017-4026.

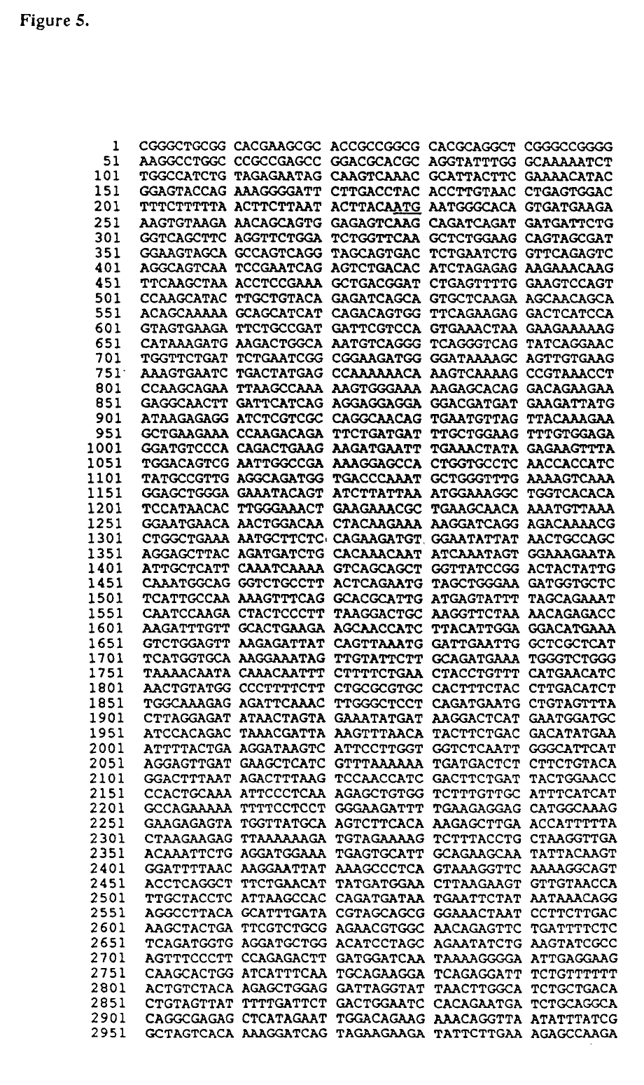

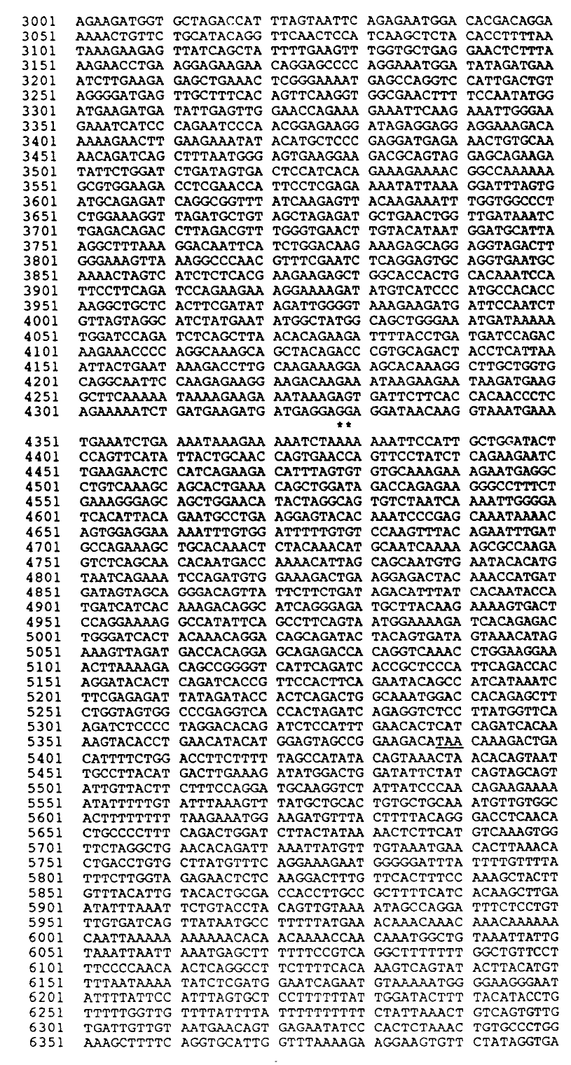

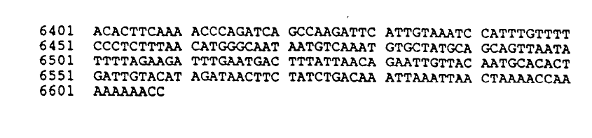

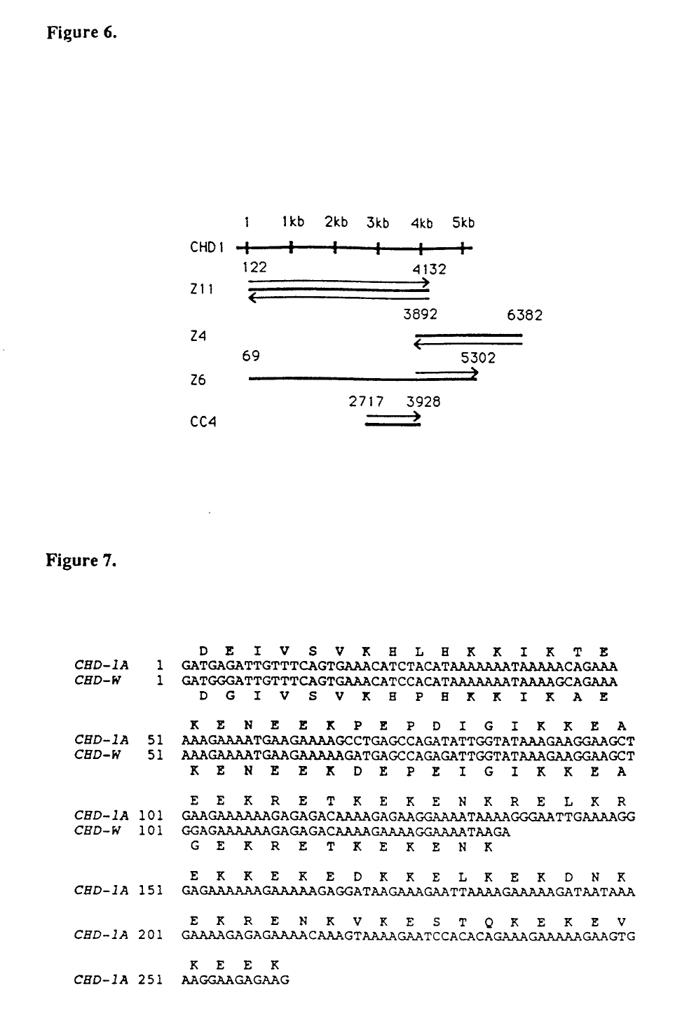

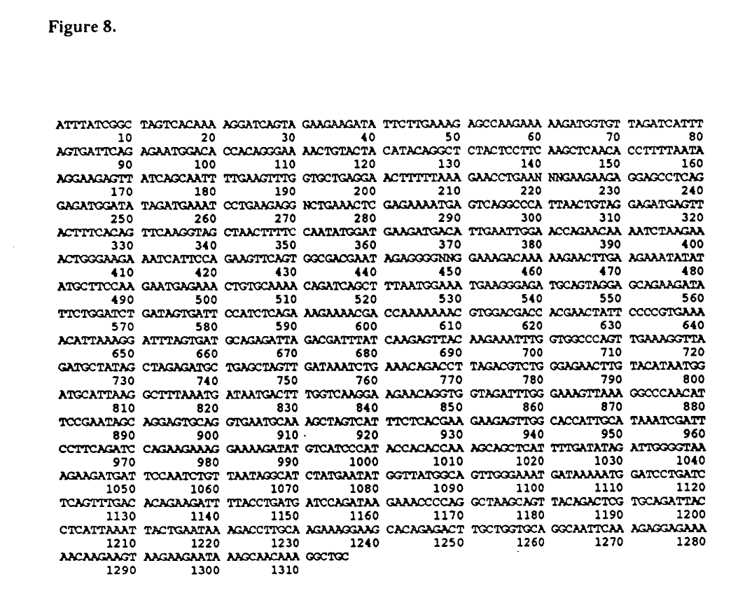

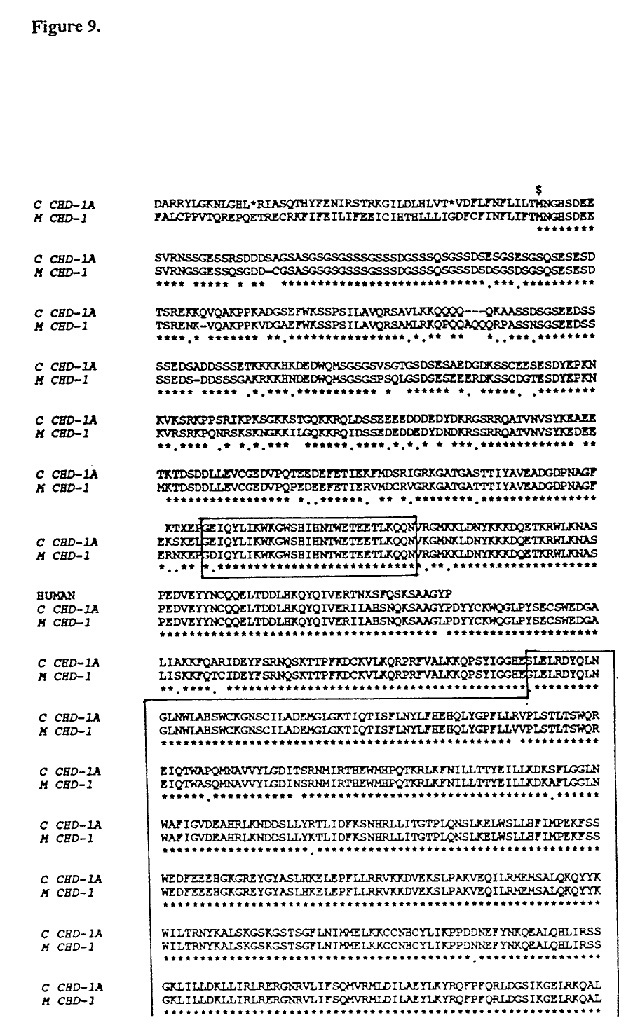

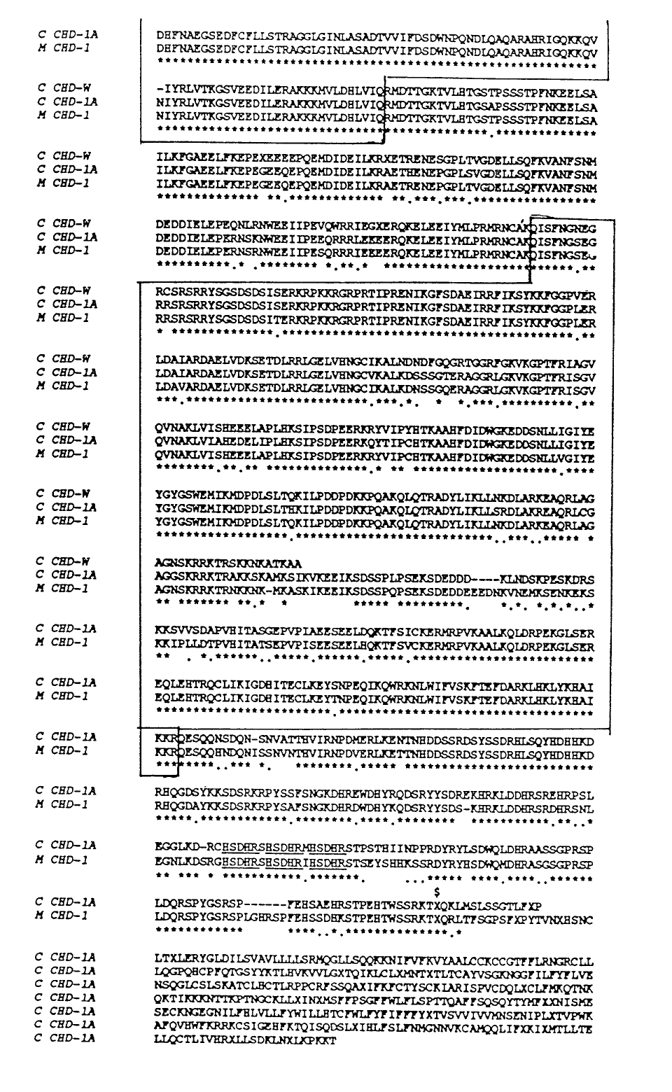

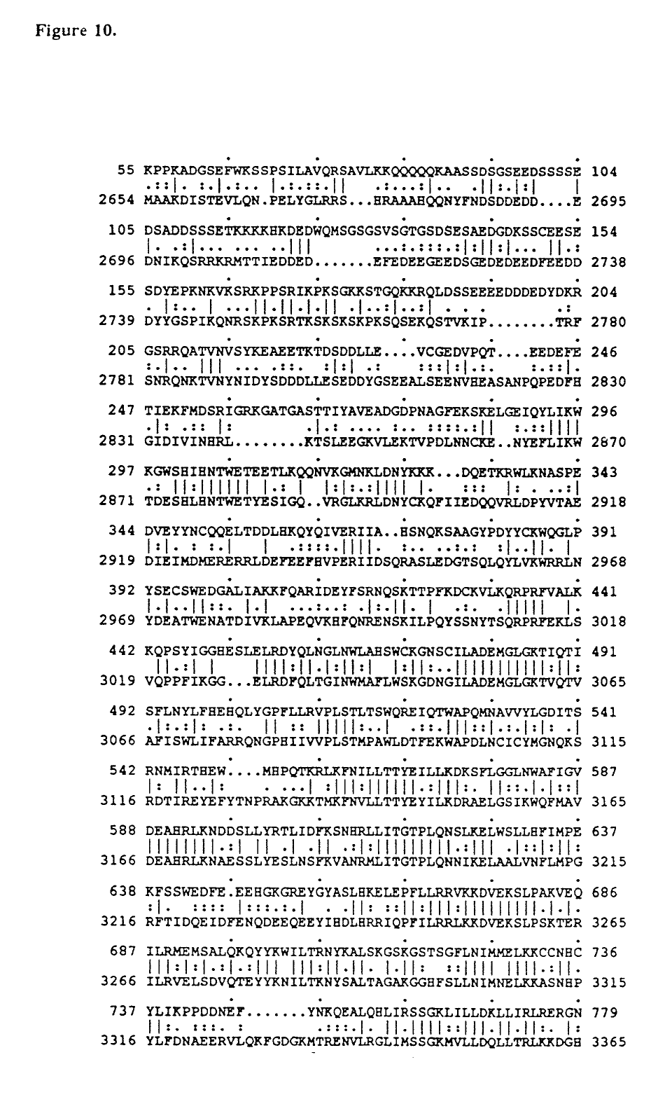

1. The nucleotide sequences of CHD-1A and CHD-W as shown in FIG. 5, FIG. 7 and FIG. 8.

2. A clone or subclones of CHD-1A and CHD-W as defined in 1.

3. A fragment of CHD-1A and CHD-W, as shown in FIG. 5, FIG. 7 and FIG. 8, capable of giving W specific signal on hybridization to genomic DNA of a non-ratite bird.

4. A fragment of CHD-1A and CHD-W, as shown in FIG. 5, FIG. 7 and FIG. 8, obtainable by restriction endonuclease digestion thereof and being capable of giving a W specific signal on hybridization to genomic DNA of a non-ratite bird.

5. A clone or subclone of a fragment according to either of claims 3 and 4.

6. A nucleic acid or fragment or oligonucleotide having substantially the sequence of CHD-1A and CHD-W as set out in FIG. 5, FIG. 7 and FIG. 8.

7. A clone or a subclone of a nucleic acid or fragment or oligonucleotide according to claim 6.

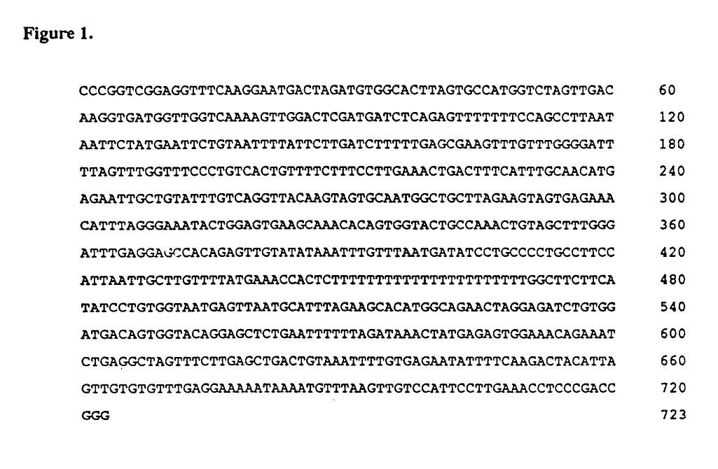

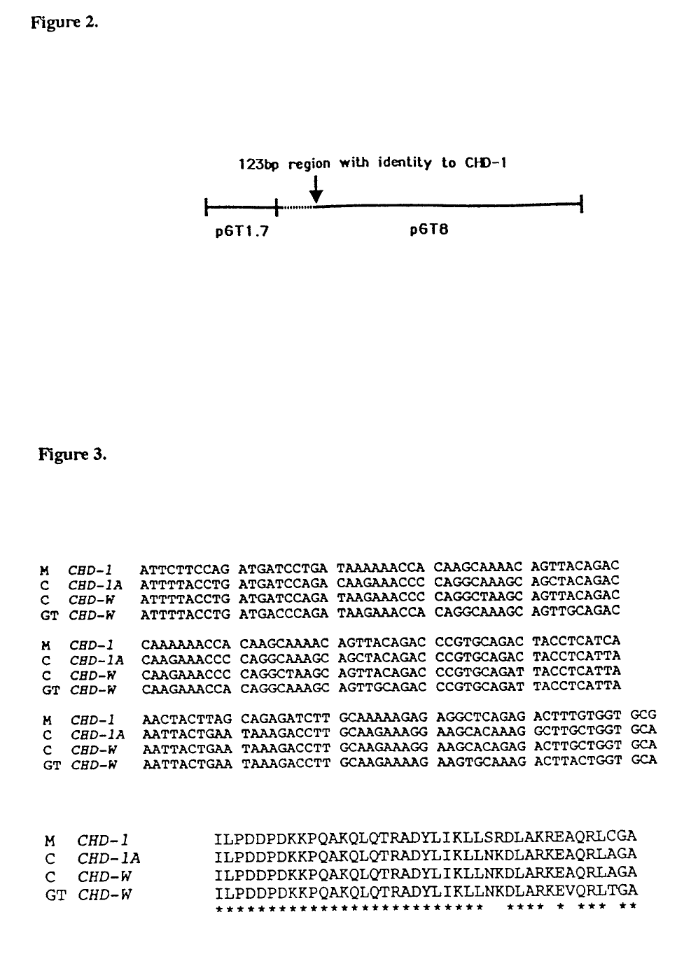

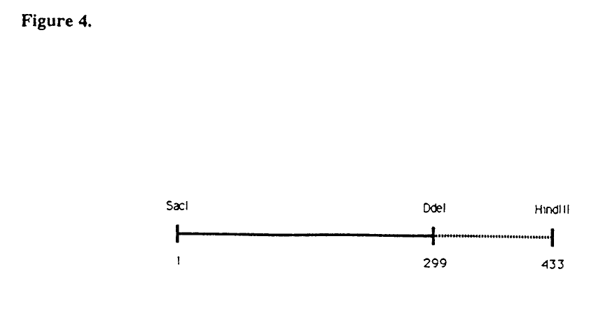

8. A nucleic acid or fragment or oligonucleotide having substantially the same sequence of the chicken or great tit CHD-gene as set out in FIG. 1, 3, 5, 7 or 8.

9. A nucleic acid or fragment or oligonucleotide containing substantially the nucleotide sequence set out in FIG. 5, FIG. 7 or FIG. 8, being capable of giving a W chromosome specific signal on hybridization to the genomic DNA of a non-ratite bird.

10. A nucleic acid or fragment or oligonucleotide according to claim 4 or claim 9 capable of giving W chromosome specific signal on hybridization to the genomic DNA of a chicken, turkey, duck, parrot.

11. A nucleic acid or fragment or oligonucleotide according to any one of claims 4, 9 and 10 capable of giving W chromosome specific signal on hybridization to the genomic DNA of a non-ratite bird under conditions of high stringency.

12. A nucleic acid or fragment or oligonucleotide according to any one of claims 4, 9 and 10 capable of giving W chromosome specific signal on hybridization to the genomic DNA of a non-ratite bird under conditions of low stringency.

13. A nucleic acid or fragment or oligonucleotide according to any one of the claims 9 to 12 containing substantially the sequence of the chicken CHD-gene as set out in FIG. 5, FIG. 7 and FIG. 8.

14. A nucleic acid or fragment or oligonucleotide, containing substantially the nucleotide sequence shown in FIG. 5, FIG. 7 or FIG. 8, encoding a CHD-protein, fragment thereof or polypeptide containing a CHD-gene or part thereof or encoding a CHD-mimetope protein or fragment thereof or CHD-mimetope polypeptide.

15. A process for ascertaining the sex of a non-ratite bird or of an embryo, foetus, cell or tissue of a non-ratite bird comprising hybridizing a nucleic acid or fragment or oligonucleotide according to any one of claims 1 to 14 with DNA or RNA of the non-ratite bird or the embryo, foetus, cell or tissue or with cDNA reverse transcribed from RNA of the non-ratite bird or the embryo, foetus, cell or tissue or with cDNA or DNA amplified by cloning or polymerase chain reaction from DNA or RNA of the non-ratite bird or the embryo, foetus, cell or tissue.

16. Use of a nucleic acid or fragment or oligonucleotide of any one of claims 1 to 14 in ascertaining the sex of a non-ratite bird or of an embryo, foetus, cell or tissue of a non-ratite bird.

17. A process for controlling the sex of the progeny of a non-ratite bird comprising inserting a nucleic acid or fragment or oligonucleotide of any one of claims 1-14 into the genome of the non-ratite_bird or progenitor thereof.

18. Use of a nucleic acid or fragment or oligonucleotide of any one of claims 1 to 14 in controlling the sex of the progeny of a non-ratite bird.

19. An avian CHD-protein, fragment thereof or polypeptide containing a CHD-gene of part thereof or a CHD-mimetope protein, fragment thereof or a CHD-mimetope polypeptide derived or derivable from an avian CHD gene.

20. A protein or fragment thereof, according to claim 19, which contains a CHD-chromobox and is derived or derivable from the nucleic acid sequence shown in FIG. 5, FIG. 7 or FIG. 8.

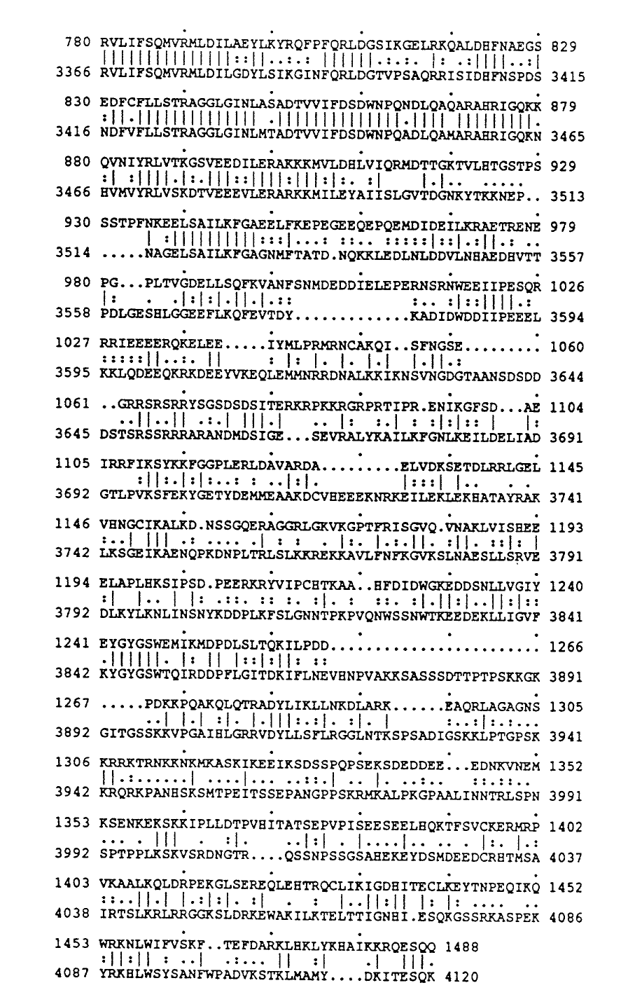

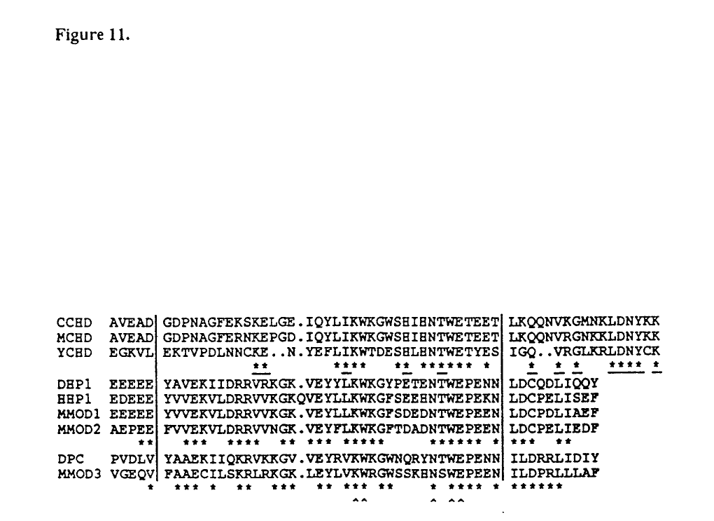

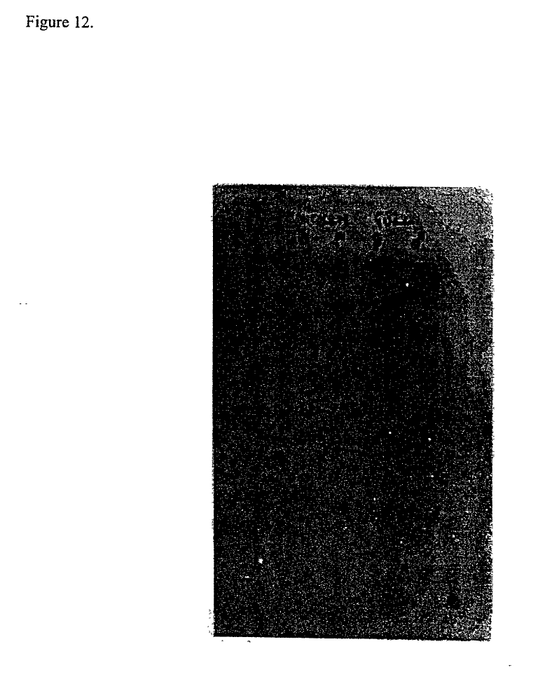

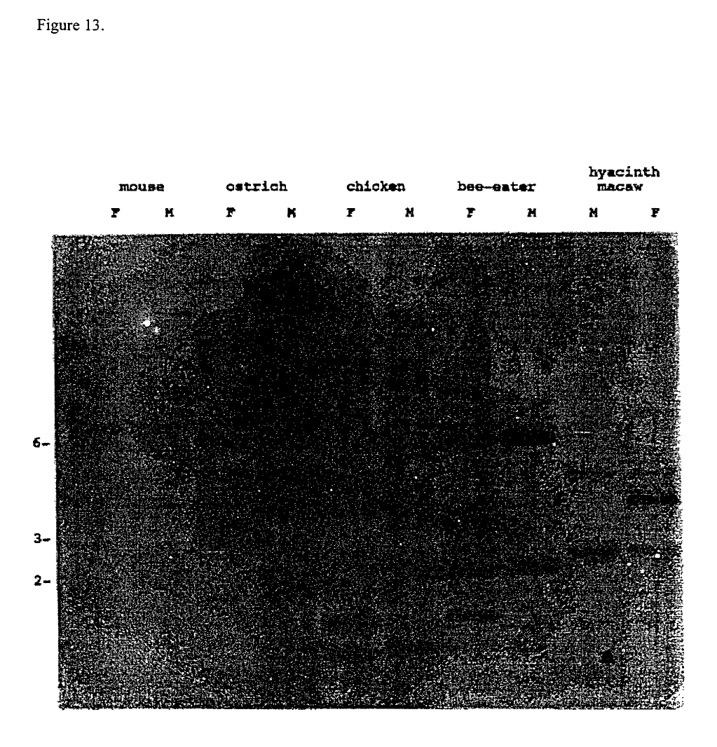

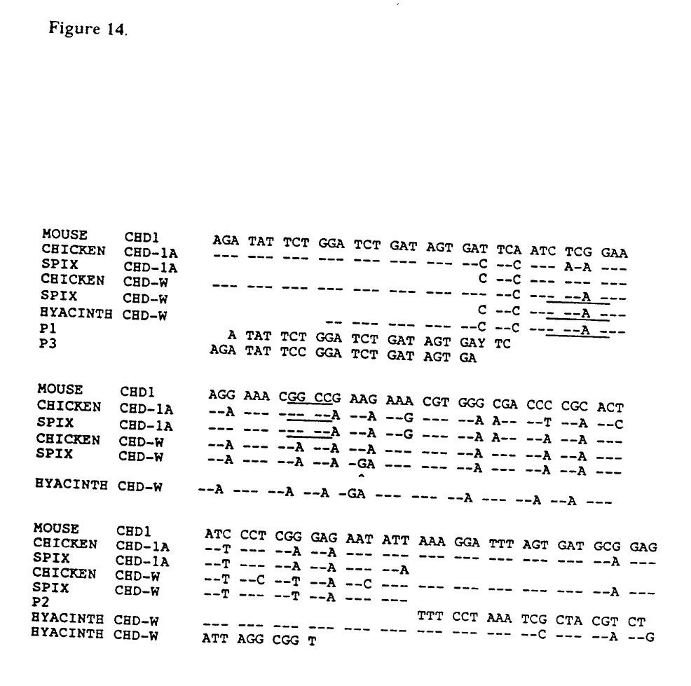

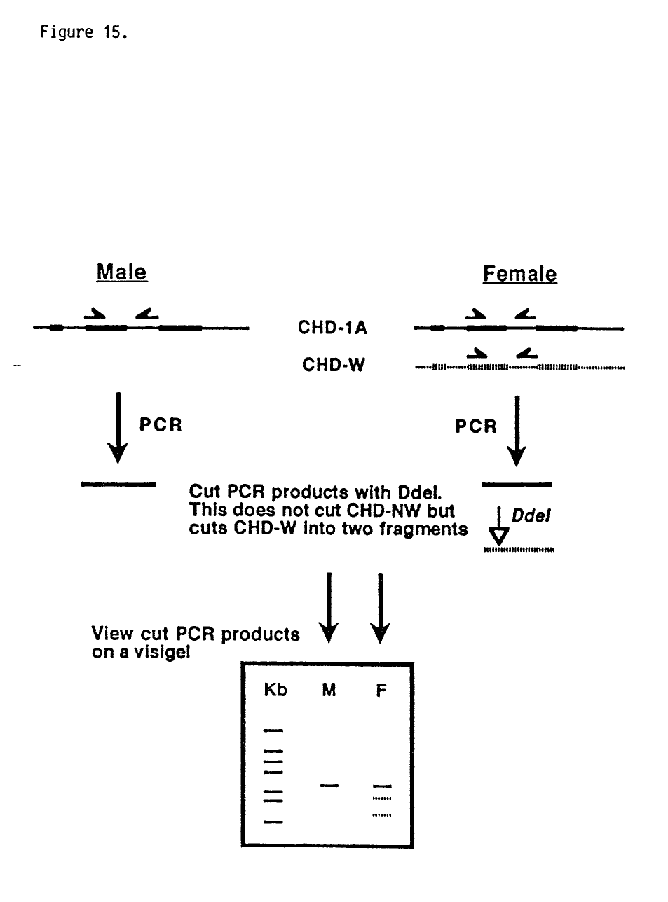

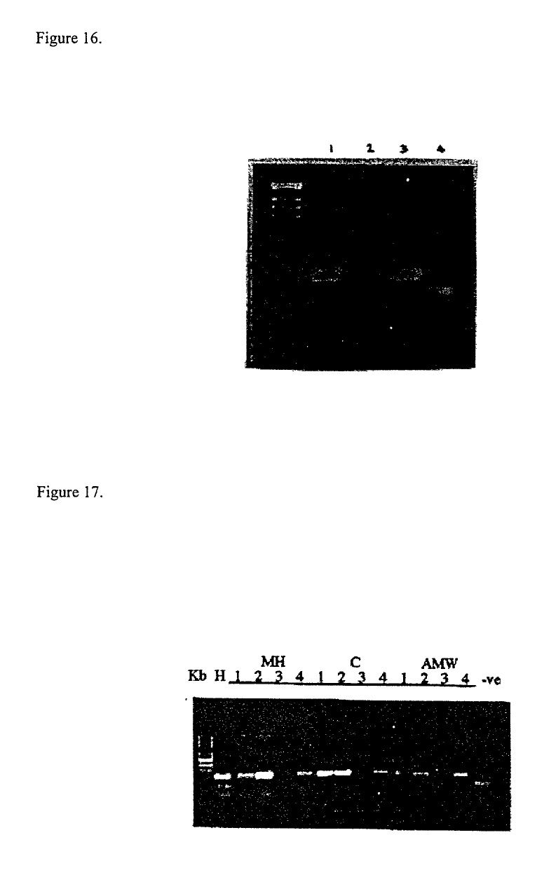

21. A protein or fragment thereof or polypeptide containing a CHD-chromobox which is derived or derivable from the nucleic acid sequence shown in FIG. 5, FIG. 7 or FIG. 8, including at least one of the characteristic amino acid residues at position 11, 12, 20, 27 or 31 inside the chromobox or 3, 6, 8, 12-15 or 16 directly downstream of the chromobox when aligned to best effect and as set out in FIG. 11.

22. A CHD-protein or fragment thereof or a polypeptide encoded by a nucleic acid or fragment or oligonucleotide according to claims 1-14 which is derived or derivable from the nucleic acid sequence shown in FIG. 5, FIG. 7 or FIG. 8 and contains a CHD-chromobox

23. A process for controlling the sex of the progeny of a non-ratite bird comprising supplying exogenously to a cell of the bird or a progenitor of the bird a protein or fragment thereof or a polypeptide according to any one of claims 19-22

24. A process according to claim 23 wherein the protein or fragment thereof or polypeptide is supplied and activates a CHD-1A or CHD-W target gene.

25. An antibody or fragment thereof against a protein or fragment thereof or polypeptide according to any one of claims 19-22.

26. An antibody producing cell capable of expressing an antibody or fragment thereof according to claim 25.

27. Use of a protein or fragment thereof or polypeptide according to any one of claims 19-22 or antibody or fragment thereof or cell according to claims 25 or 26 in ascertaining the sex of an embryo cell or tissue of a non-ratite bird.

28. A transgenic or chimeric non-ratite bird having a heterologous nucleic acid or fragment or oligonucleotide according to any one of claims 1 to 14 in the genome of at least the germ cells of the bird.

29. Gametes of a non-ratite bird according to claim 28.

30. Progeny of a non-ratite bird according to claim 28.

31. Progeny according to claim 30 which are transgenic or chimeric and have a heterologous nucleic acid or fragment according to any one of claims 1-14 in the genome of at least the germ cells of the progeny.

32. A method of controlling the population of a species of bird which comprises introducing an individual member of the species into the population, said individual having a copy or copies of a nucleic acid fragment or oligonucleotide according to any one of claims 1 to 14 integrated on a chromosome (carrier chromosome) be it sex linked or autosomal whereby when the male breeds with other individuals of the population the progeny are substantially of one sex or are sexually dysfunctional intersexes.

33. A method according to claim 32 where the nucleic acid integrated into the carrier chromosome is homologous to the native CHD-1A or CHD-W gene of the bird.
